(19) United States
(12) Patent Application Publication
(10) Pub. No.: US 2016/0361026 A1
(43) Pub. Date: Dec. 15, 2016

(54) METHOD AND APPARATUS FOR MONITORING TISUE FLUID CONTENT FOR USE IN AN IMPLANTABLE CARDIAC DEVICE

(71) Applicants: Shantanu Sarkar, Minnesota, MN (US); Douglas A. Hettrick, Andover, MN (US); Amul Y. Desai, Plymouth, MN (US); Randolph M. Biallas, Littleton, CO (US); Holly S. Vitense, Maple Grove, MN (US); Jodi L. Redemske, Fridley, MN (US)

(72) Inventors: Shantanu Sarkar, Minnesota, MN (US); Douglas A. Hettrick, Andover, MN (US); Amul Y. Desai, Plymouth, MN (US); Randolph M. Biallas, Littleton, CO (US); Holly S. Vitense, Maple Grove, MN (US); Jodi L. Redemske, Fridley, MN (US)

(73) Assignee: Medtronic, Inc., Minneapolis, MN (US)

(21) Appl. No.: 13/391,376

(22) PCT Filed: Mar. 29, 2011
(86) PCT No.: PCT/US2011/030268

§ 371 Date: Feb. 20, 2012

Related U.S. Application Data
(60) Provisional application No. 61/318,588, filed on Mar. 29, 2010.

(51) Int. Cl. A61B 5/00; A61B 5/0245; G06F 19/00; A61N 1/365; A61N 1/362; A61B 5/024; A61B 5/053

(52) U.S. Cl. CPC A61B 5/7275; A61B 5/02405; A61B 5/0245; A61B 5/0538; A61B 5/686; A61N 1/36521; A61N 1/3627; A61B 5/742; A61B 5/7264; G06F 19/3431; G06F 19/345

(57) ABSTRACT

Techniques for using multiple physiological parameters to provide an early warning for worsening heart failure are described. A system is provided that monitors a multiple diagnostic parameters indicative of worsening heart failure. The parameters preferably include are least one parameter acquired from an implanted device, such as intrathoracic impedance. The system device derives an index of the likelihood of worsening heart failure based upon the parameters using a Bayesian approach and displays the resultant index for review by a physician.

### CROSS-REFERENCE TO RELATED APPLICATION

This is a Section 371 National Stage Application of International No. PCT/US2011/030268, filed on Mar. 29, 2011, and published as WO 2011/126823 A1 on Oct. 13, 2011, which claims priority from U.S. Patent Application No. 61/318,588, filed 29 Mar. 2010, the contents of which are incorporated herein in their entirety for all purposes.

### FIELD OF THE INVENTION

The invention relates to medical devices and, more particularly, devices for the diagnosis of worsening heart failure and treatment of related ailments.

### BACKGROUND OF THE INVENTION

A variety of medical devices have been used or proposed for use to deliver a therapy to and/or monitor a physiological condition of patients. As examples, such medical devices may deliver therapy and/or monitor conditions associated with the heart, muscle, nerve, brain, stomach or other organs or tissue. Medical devices that deliver therapy include medical devices that deliver one or both of electrical stimulation or a therapeutic agent to the patient. Some medical devices are implantable medical devices (IMDs) that are implanted within the patient.

Some medical devices have been used or proposed for use to monitor heart failure or to detect heart failure events. Typically, such medical devices have been implantable and, in many cases, have been cardiac pacemakers, cardioverters and/or defibrillators with added heart failure monitoring functionality. In some cases, such medical devices have monitored heart failure by monitoring intrathoracic impedance, which may provide a good indication of the level of edema in patients. While edema is a sign of many other conditions it is also a sign of worsening heart failure. Worsening heart failure may result in cardiac chamber dilation, increased pulmonary blood volume, and fluid retention in the lungs—all of which contribute to a decrease in intrathoracic impedance. Other diagnostic parameters, such as heart rate variability, have been proposed for use in such devices to identify worsening heart failure or heart failure events.

Generally, the first indication that a physician would have of the occurrence of edema in a patient is not until it becomes a physical manifestation with swelling or breathing difficulties so overwhelming as to be noticed by the patient who then proceeds to be examined by a physician. This is undesirable since hospitalization at such a time would likely be required for a heart failure patient. Accordingly, medical devices have been used to monitor impedance in patients and provide an alert to the patient to seek medical treatment prior to the onset of worsening heart failure with symptoms, such as edema, that require hospitalization.

A variety of approaches to diagnosis of impending heart failure (HFH) have been proposed. Heart rate variability (HRV), activity, and night heart rate (NHR) have been shown to be predictive of HFH1. Adamson and colleagues demonstrated that HRV and activity decreased and NHR increased prior to HFH. See 1 Adamson P B, Smith A L, Abraham W T, Kleckner K J, Stadler R W, Shih A, Rhodes M M; InSync III Model 8042 and Attain OTW Lead Model 4193 Clinical Trial Investigators. Continuous autonomic assessment in patients with symptomatic heart failure: prognostic value of heart rate variability measured by an implanted cardiac resynchronization device, Circulation. 2004 Oct. 19;110(16):2389-94. Heart rate variability (SDAAM), activity and night heart rate are also predictive of heart failure hospitalizations.

A multi-parameter approach for predicting heart failure is described in U.S. patent application Ser. No. 12/184,003 by Sarkar, et al. for “Using Multiple diagnostic parameters for predicting heart failure events”, filed Jul. 31, 2008 and incorporated herein by reference in its entirety. This approach combines the alert rate reducing capabilities of increasing the threshold with the Heart Failure (HF) prediction capabilities of the other diagnostic parameters in a typical device.

Applying the multi-parameter algorithm of the Sarkar, et al application on the fluid index of OptiVol 1.0 resulted in a 39% reduction in false positives without losing any true positive detection in the OFISSER study. See Small R, Wickemeyer W, Germany R, Hoppe B, Andriulli J, Brady P, LaBeau M, Sarkar S, Hettrick D A Tang W; Changes in Intrathoracic Impedance are Associated with Subsequent Risk of Hospitalizations for Acute Decompensated Heart Failure: Clinical Utility of Implanted Device monitoring without a Patient Alert. Journal of Cardiac Failure, 2009, incorporated herein by reference in its entirety.

The Optivol fluid index referred to herein corresponds to the correspondingly named feature in commercially available Medtronic devices. Descriptions of this feature and of enhancements thereto can be found in U.S. Pat. No. 6,512,949, issued to Combs, et al. on January 26, 2003 and US Patent Publications US2008/0027349A1 and US2008/0024293A1, by Stylos, published Jan. 31, 2008, all assigned to Medtronic Inc and incorporated herein by reference in their entireties.

Although quite promising, the “multi-parameter” model described above includes only device diagnostics. Thus, it is currently unknown whether a model that combines both device and non-device information into a single parameter could further improve the accuracy and clinical utility of a derived heart failure index.

### BRIEF SUMMARY OF THE INVENTION

The present invention is intended for use in the context of a device system generally as disclosed in U.S. patent application Ser. No. 12/184,003 by Sarkar, et al. for “Using Multiple diagnostic parameters for predicting heart failure events”, filed Jul. 31, 2008 and incorporated herein by reference in its entirety. The diagnostic analysis system should be understood to be embodied in a system as disclosed in the cited application, incorporated in conjunction with or as an alternative to the diagnostic capabilities of the system disclosed therein. The hardware employed in the system, including the implanted devices from which diagnostic data is gathered, should be understood to correspond to the hardware disclosed in the cited application. Similarly, the programmer for the implanted devoice, the access point to the network, the network to which the implantable device is connected and the external device servers and computing devices coupled to the network all correspond generally to those described in the cited application, with the exceptions of the newly added features of the present invention as discussed in detail below

The results of the analytical methodology described below should be understood to be displayed on the programmer and/or the server and computing devices coupled to the network, for review of the physician in the same general manner as discussed in the cited Sarkar, et al. application. A summary description of the corresponding system and devices therein is included below.

In addition to device derived diagnostic parameters, many external home monitored diagnostics including weight, blood pressure and patient symptoms, are useful for evaluating patients with heart failure. Patient self-care, or “adherence” to prescribed pharmacologic regiments, dietary restrictions and physical activity recommendations also impact heart failure signs and symptoms. The present invention is directed to usefully employing patient monitored signs and symptoms to augment current heart failure monitoring parameters from an implantable device, such as intrathoracic impedance, heart rate variability or filling pressure.

In furtherance of this desired result, the inventors have developed and validated of an HF risk score based on multiple device and non device parameters within the Bayesian Belief Network modeling framework. The goal of this risk score is to provide a single integrated heart failure parameter that indicates the probability of HF hospitalization in the near term. Such an index should also have important clinical utility for identifying less severe signs and symptoms of worsening heart failure and poor adherence.

In contrast to the OptiVol fluid index provided by pacemakers currently, available from Medtronic, Inc., sold the resultant “HF risk score” is a continuous variable indicating relative degrees of risk and severity of worsening heart failure events.

The invention provides a multi-parameter heart failure index including both device and non-device gathered parameters. The invention was developed using Bayesian Belief network modeling theory. The development data set included several existing clinical trials including OFISSER, The OptiVol clinical Case Studies, the Italian Clinical Services and the CONNECT trial (interim freeze). The validation set consisted of the PARTNERS and FAST trial data. The algorithm definition process involved the application of the development data sets to identify the critical baseline parameters, define prior probabilities and define likelihood tables.

A Bayesian Belief Network table was generated based on prior probabilities and likelihood tables. At each evaluation, the previous 30 days of data were searched and two different risk scores including the maximum of daily HF risk scores and the monthly HF risk score were computed. The presence of an event in the 30 days following evaluation was considered for statistical analysis. In order to avoid the presumption of an alert based index, metrics such as sensitivity and PPV, and false alert rates were not evaluated. Rather, a Generalized Estimating Equation model was used to evaluate the results in a monthly evaluation usage simulation. The HF risk score was divided into quartiles and the odds ratio and probability of event for each of the quartiles were reported for each study.

The baseline parameters demonstrating statistical contribution to the model included a history of hypertension, atrial fibrillation, diabetes as well as NYHA class, ejection fraction <35%, QRS duration=120 ms, and CRT (vs. ICD) device. Device Parameters incorporated in the model included the OptiVol fluid index, activity, night heart rate, heart rate variability, VT/VF, AT/AF, ventricular rate during AT/AF and % Ventricular Pacing. Non Device Parameters incorporated in the model included HF hospitalization or symptoms, weight, blood pressure and medication adherence. The entire development set consisted of 921 patients with 9790 monthly evaluations with 104 monthly evaluations having at least one event. Similarly, the entire validation set consisted of 784 patients with 8149 monthly evaluations with 122 monthly evaluations having at least one event.

The integrated diagnostics approach of the present provides a single parameter at any investigation that indicates the probability of HF hospitalization in the future. The HF risk score provided is a continuous variable with a higher value indicating a higher risk.

### BRIEF DESCRIPTION OF THE DRAWINGS

FIG. 1 is a graph showing a comparison of raw heart failure events for all 4 quartiles of HF risk for both the development and validation data sets.

FIG. 2 is a graph illustrating a case example of smoothed HF risk score for one patient.

FIG. 3 is a graph illustrating the Impact of removing OptiVol from the model as well as impact of running the model with only OptiVol:

FIG. 4 is a conceptual diagram illustrating an example of a system that may be used to detect worsening heart failure in patient 14 using the methodology of the present invention.

FIG. 5 is a diagram illustrating an implantable device system of the type which may be used in conjunction with the present invention.

FIG. 6 is a functional block diagram of one example of IMD 16, illustrated in FIG. 4.

FIG. 7 is a block diagram of an example programmer 24, illustrated in FIG. 4.

FIG. 8 illustrates an example of a simple Bayesian Belief Network (BBN)

FIG. 9 is a conditional probability table.

FIG. 10 is a simplified conditional probability table.

FIG. 11 is a functional illustration of the generation of the BBN probability tables.

FIG. 12 is a functional illustration of the over-all methodology of generation of the HF risk scores according to the present invention.

FIG. 13 is a table setting forth a categorization of baseline probabilities.

FIG. 14 is a table setting forth values for activity, NHR and HRV computations.

FIG. 15 is a table setting forth the criteria state mapping for the device variables

FIG. 16 is a table setting forth the criteria state mapping for the clinical variables.

FIG. 17 is an illustration of a progression of the HF risk score over time

FIG. 18 is a graph illustrating the inter-relation of the HF risk score to other diagnostic variables in a patient.

FIG. 19 is a table of the distribution of the HFH events among the development database.

FIG. 20 is a table setting forth usage of the various clinical studies relied upon to develop and validate the methodology of the present invention

FIG. 21 is a time line illustrating the evaluation methodology for the diagnostic score provided by the present invention.

FIG. 22 baseline characteristics for patients from the CONNECT study.

FIG. 23 is a table setting forth the univariate logistic regression of baseline variables vs. presence or absence of HF events from the CONNECT study.

FIG. 24 is a table setting forth the multivariate logistic regression of baseline variables vs. presence or absence of HF events from the CONNECT study.

FIG. 25 is a likelihood table for the Opti-Vol criteria states.

FIG. 26 is a likelihood table for the Activity criteria states.

FIG. 27 is a likelihood table for the NHR criteria states.

FIG. 28 is a likelihood table for the HRV criteria states.

FIG. 29 is a likelihood table for the Combination criteria states.

FIG. 30 is a likelihood table for the Event/Symptom criteria states.

FIG. 31 is a likelihood table for the Weight criteria states.

FIG. 32 is a table setting forth the HF Risk Score logistic regression results for the development data set.

FIG. 33 is a table setting forth the HF Risk Score divided into Quartiles for the development data set.

FIG. 34 is a graph illustrating the distribution of events in the four quartiles of HF Risk Score of the development data set.

FIG. 35 is a table setting forth the HF Risk Score logistic regression results for the validation data set.

FIG. 36 is a table setting forth is a table setting forth the Risk Score divided into Quartiles for the validation data set.

FIG. 37 is a graph illustrating the distribution of events in the four quartiles of HF Risk Score of the validation data set.

FIGS. 38-41 are display screens illustrating a display methodology for use in conjunction with the HF Risk Score.

### DETAILED DESCRIPTION OF THE PREFERRED EMBODIMENTS

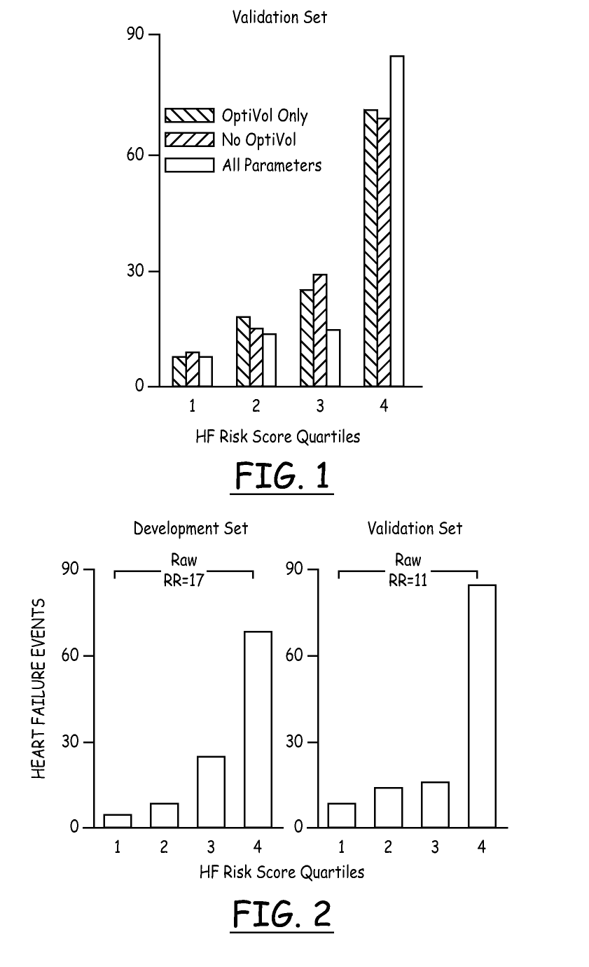

FIG. 1 shows a comparison of raw heart failure events for all 4 quartiles of HF risk for both the development and validation data sets. The results of the GEE adjusted logistic regression analysis revealed that months with a maximum of daily or overall HF risk score in the highest quartile were much more likely to experience a HF event. As illustrated in FIG. 1. This result was highly statistically significant for both the design and validation data sets. For both data sets, the risk of the HF event increased significantly as the HF risk score increased.

FIG. 2 is a graph illustrating a case example of smoothed HF risk score for one patient. The high risk period was associated with an HF hospitalization. The next highest risk period was not associated with Hospitalization but was associated with reported worsening signs and symptoms.

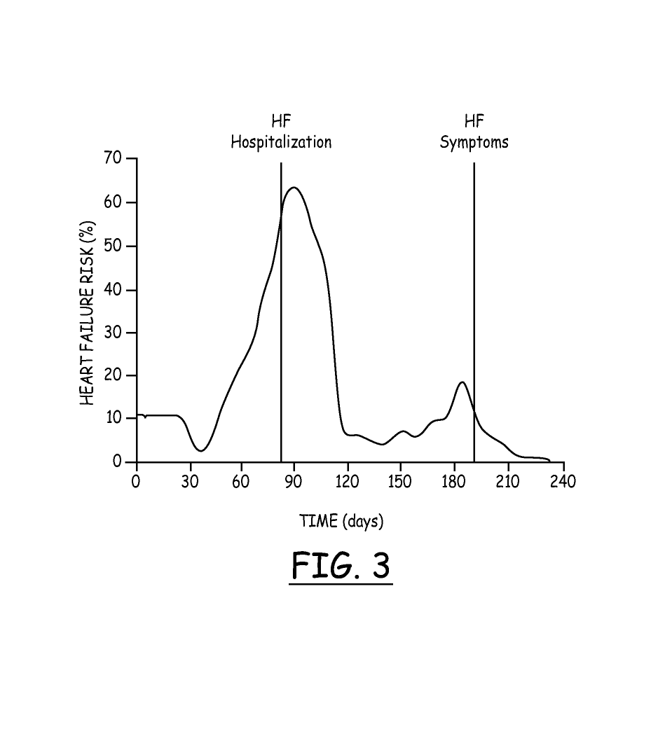

FIG. 3 is a graph illustrating the: Impact of removing OptiVol from the model as well as impact of running the model with only OptiVol: This figure demonstrates that all model components are important in order to maximize model performance. An OptiVol only model or a model excluding OptiVol would have inferior performance. FIG. 3 demonstrates that running the model with only OptiVol or conversely, with everything but OptiVol, negatively impacts the validation set results.

The invention as disclosed in more detail below thus generated a continuous absolute HF risk score that appropriately identifies time periods with clinically and statistically significantly higher relative risk for near term HF clinical events. Conversely, the index also identifies a group at relatively low risk for events. This HF score provided by the invention is thus believed superior to a single parameter approach, for example OptiVol, or to an (“apples and apples”) Bayesian approach using only a single parameter.

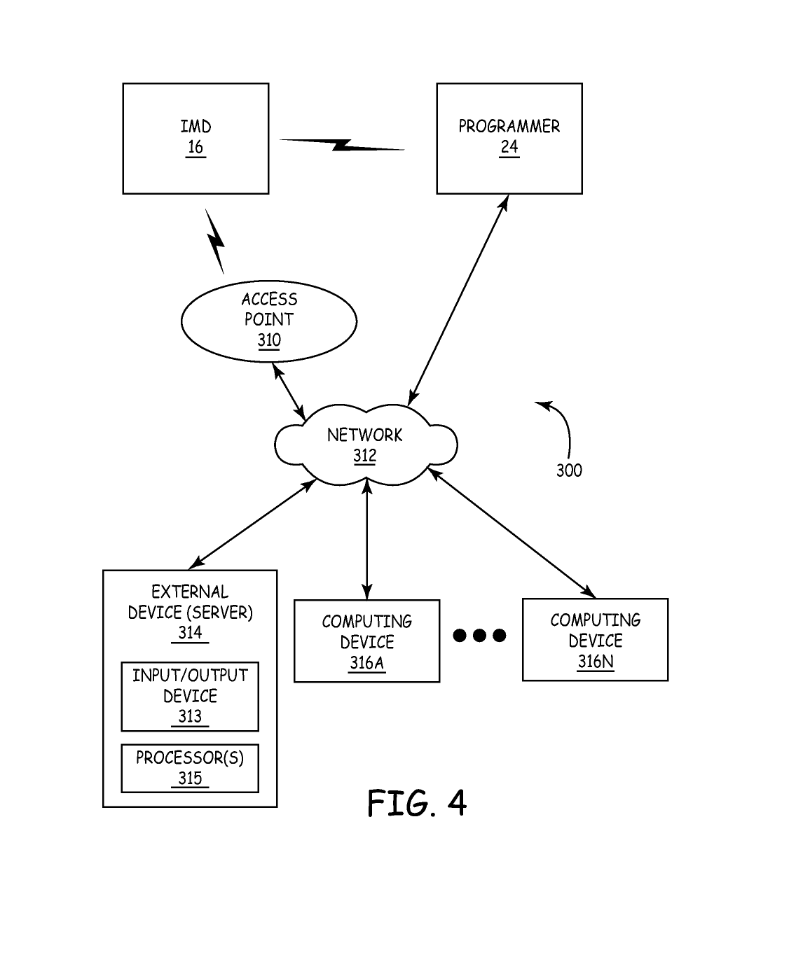

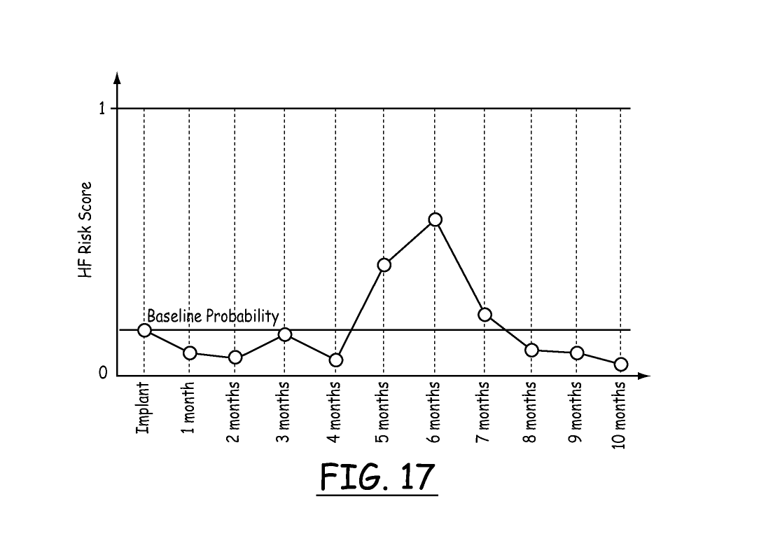

FIG. 4 is a block diagram illustrating an example system 300 that includes an external device, such as a server 314, and one or more computing devices 316A-316N (“computing devices 316”) that are coupled to IMD 16 and programmer 24 via a network 312. In this example, IMD 16 may use its telemetry module 88 to communicate with programmer 24 via a first wireless connection, and to communication with an access point 310 via a second wireless connection. In the example of FIG. 17, access point 310, programmer 24, server 314, and computing devices 316A-216N are interconnected, and able to communicate with each other, through network 312. In some cases, one or more of access point 310, programmer 24, server 314, and computing devices 316A-316N may be coupled to network 312 through one or more wireless connections. IMD 16, programmer 24, server 314, and computing devices 316A-216N may each comprise one or more processors, such as one or more microprocessors, DSPs, ASICs, FPGAs, programmable logic circuitry, or the like, that may perform various functions and operations, such as those described herein. For example, as illustrated in FIG. 17, server 314 may comprise one or more processors 315 and an input/output device 313, which need not be co-located.

Server 314 may, for example, monitor primary and secondary diagnostic parameters, e.g., based on signals or information received from IMD 16 and/or programmer 24 via network 312, to detect worsening heart failure of patient 14 using any of the techniques described herein. Server 314 may provide alerts relating to worsening heart failure of patient 16 via network 312 to patient 14 via access point 310, or to one or more clinicians via computing devices 316. In examples such as those described above in which IMD 16 and/or programmer 24 monitor the primary and secondary diagnostic parameters, server 314 may receive an alert from the IMD or programmer via network 312, and provide alerts to one or more clinicians via computing devices 316. Server 314 may generate web-pages to provide alerts and information regarding the primary and secondary diagnostic parameters, and may comprise a memory to store alerts and diagnostic or physiological parameter information for a plurality of patients.

Access point 310 may comprise a device that connects to network 312 via any of a variety of connections, such as telephone dial-up, digital subscriber line (DSL), or cable modem connections. In other embodiments, access point 310 may be coupled to network 312 through different forms of connections, including wired or wireless connections. Network 312 may comprise a local area network, wide area network, or global network, such as the Internet. System 300 may be implemented, in some aspects, with general network technology and functionality similar to that provided by the Medtronic CareLink® Network developed by Medtronic, Inc., of Minneapolis, Minn.

Additionally, using programmers 24, access points 310 or computing devices 316, physicians and/or event patients may input clinical information regarding the patients (such as symptoms, lab results, health care utilizations, etc.) that may be used as secondary parameters by the heat failure risk quantification methodology as described herein. Alternatively or in addition, the present methodology of the present invention with respect to monitoring worsening heart failure may be provided by any one or more of the programmers 24, access points 310, server 314, or computing devices 316. Similarly, the functions included within the methodology of the present invention may be distributed between these devices, as desired.

In the specific embodiment discussed herein, the method of the present invention is embodied in software associated with server 314, which, as discussed above, monitors the primary and secondary diagnostic parameters, e.g., based on signals or information received from IMD 16 and/or programmer 24 via network 312, to calculate the HF risk score provided by the present invention and provide this information to the physician for display using the other devices as described above.

The techniques described in this disclosure, including those attributed to image IMD 16, programmer 24, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. Although described primarily with reference to intrathoracic impedance, in some examples a cardiovascular pressure may additionally or alternatively be used as a primary diagnostic parameter. In some examples, a fluid index may increase based on increasing cardiovascular pressure over time, in a substantially similar manner to that which the fluid index discussed above increased based on decreasing intrathoracic impedance over time. Examples of cardiovascular pressures that may be monitored are right ventricular pressure, left atrial pressure, or estimated pulmonary artery diastolic pressure.

Furthermore, although described primarily with reference to examples that provide a metric related to the risk of worsening heart failure, other examples may additionally or alternatively automatically modify a therapy in response to detecting worsening heart failure in the patient. The therapy may be, as examples, a substance delivered by an implantable pump, cardiac resynchronization therapy, refractory period stimulation, or cardiac potentiation therapy.

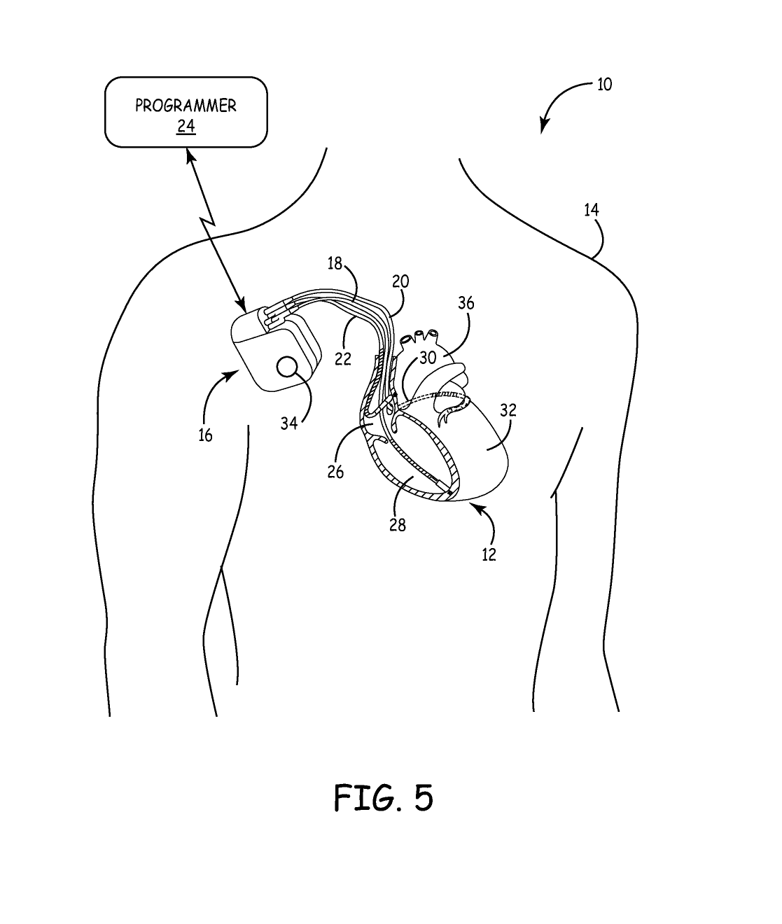

FIG. 5 is a conceptual diagram illustrating an example system 10 that may be used to detect the risk of worsening heart failure in patient 14 using the present invention. The system of FIG. 5 corresponds to the system disclosed in the above-cited Sarkar application, and is described in more detail therein.

System 10 includes implantable medical device (IMD) 16, which is coupled to leads 18, 20, and 22, an electrode 34 located on the can of IMD 16, and a programmer 24. In some examples, IMD 16 may be a purely diagnostic device that monitors multiple diagnostic parameters associated with heart failure. In other examples, IMD 16 may additionally operate as a therapy delivery device to deliver electrical signals to heart 12 via one or more of leads 18, 20, and 22, such as an implantable pacemaker, a cardioverter, and/or defibrillator. In some examples, IMD 16 may operate as a drug delivery device that delivers therapeutic substances to patient 14 via catheters (not shown), or as a combination therapy device that delivers both electrical signals and therapeutic substances. Moreover, IMD 16 is not limited to devices implanted as shown in FIG. 1. As an example, IMD 16 may be implanted subcutaneously in patient 14, or may be an entirely external device with leads attached to the skin of patient 14 or implanted percutaneously in patient 14. In some examples, IMD 16 need not include leads, but may include a plurality of electrodes, like electrode 34, on the housing of IMD 16.

In general, IMD 16 monitors a primary diagnostic parameter that is indicative of fluid accumulation and one or more secondary diagnostic parameters. In particular, IMD 16 may monitor the primary diagnostic parameter and the one or more secondary diagnostic parameters at the same time. Example, primary diagnostic parameters include intrathoracic impedance and cardiovascular pressure. Example secondary diagnostic parameters include atrial fibrillation burden (AF), heart rate during AF, ventricular fibrillation burden (VF), heart rate during VF, atrial tachyarrhythmia burden (AT), heart rate during AT, ventricular tachyarrhythmia burden (VT), heart rate during VT, activity level, heart rate variability, night heart rate, difference between day heart rate and night heart rate, heart rate turbulence, heart rate deceleration capacity, respiratory rate, baroreflex sensitivity, percentage of cardiac resynchronization therapy (CRT) pacing, metrics of renal function, weight, blood pressure, symptoms entered by the patient via a programmer, and patient history, such as medication history, or history of heart failure hospitalizations. The specific diagnostic parameters employed by the disclosed embodiment of the present invention are discussed in more detail below. Thus, IMD 16 may, in various embodiments, monitor either intrathoracic impedance or pressure and one, all, or any combination of the previously recited secondary diagnostic parameters.

In the example illustrated in FIG. 4, IMD 16 is configured to monitor intrathoracic impedance and includes leads 18, 20, and 22 extend into the heart 12 of patient 14. Right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12. Other configurations, i.e., number and position of leads, are possible. For example, other leads or lead configurations may be used to monitor pressure and various secondary diagnostic parameters. As described above, in some examples, IMD 16 need not be coupled to leads.

Intrathoracic impedance, as well as various secondary diagnostic parameters, may be measured by creating an electrical path between electrodes (not shown in FIG. 4) located on one or more of leads 18, 20, and 22 and can electrode 34. In some embodiments, the can of IMD 16 may be used as an electrode in combination with electrodes located on leads 18, 20, and 22. For example, system 10 may measure intrathoracic impedance by creating an electrical path between RV lead 18 and electrode 34. In additional embodiments, system 10 may include an additional lead or lead segment having one or more electrodes positioned at a different location in the cardiovascular system or chest cavity, such as within one of the vena cava, subcutaneously at a location substantially opposite IMD 16 vis-à-vis the thorax of patent 14, or epicardially, for measuring intrathoracic impedance.

In embodiments in which IMD 16 operates as a pacemaker, a cardioverter, and/or defibrillator, IMD 16 may sense electrical signals attendant to the depolarization and repolarization of heart 12 via electrodes coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. IMD 16 may also provide defibrillation therapy and/or cardioversion therapy via electrodes located on at least one of the leads 18, 20, 22. IMD 16 may detect arrhythmia of heart 12, such as fibrillation of ventricles 28 and 32, and deliver defibrillation therapy to heart 12 in the form of electrical pulses. In some examples, IMD 16 may be programmed to deliver a progression of therapies, e.g., pulses with increasing energy levels, until a fibrillation of heart 12 is stopped.

In some examples, programmer 24 may be a handheld computing device, computer workstation, or networked computing device. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some embodiments, a display of programmer 24 may include a touch screen display, and a user may interact with programmer 24 via the display. It should be noted that the user may also interact with programmer 24 remotely via a networked computing device.

A user, such as a physician, technician, surgeon, electrophysiologist, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD. For example, the user may use programmer 24 to retrieve information from IMD 16. The information may relate to the primary and/or secondary diagnostic parameters discussed above. In addition, the user may use programmer 24 to retrieve information from IMD 16 regarding the performance or integrity of IMD 16 or other components of system 10, such as leads 18, 20, and 22, or a power source of IMD 16.

Furthermore, the user may use programmer 24 to enter clinical information that can be used as secondary parameters, such as patient history, medication history, history of heart failure hospitalizations, or other historical or current observations of patient condition.

Programmer 24 may also be used to program a therapy progression, select electrodes to deliver defibrillation pulses, select waveforms for the defibrillation pulse, or select or configure a fibrillation detection algorithm for IMD 16. In particular, the physician may use programmer 24 to adjust the therapies provided by the IMD as appropriate, responsive to the HF Risk Score and associated information as provided using the display methodology described below.

The user may also use programmer 24 to program aspects of other therapies provided by IMD 16, such as cardioversion or pacing therapies. In some examples, the user may activate certain features of IMD 16 by entering a single command via programmer 24, such as depression of a single key or combination of keys of a keypad or a single point-and-select action with a pointing device.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

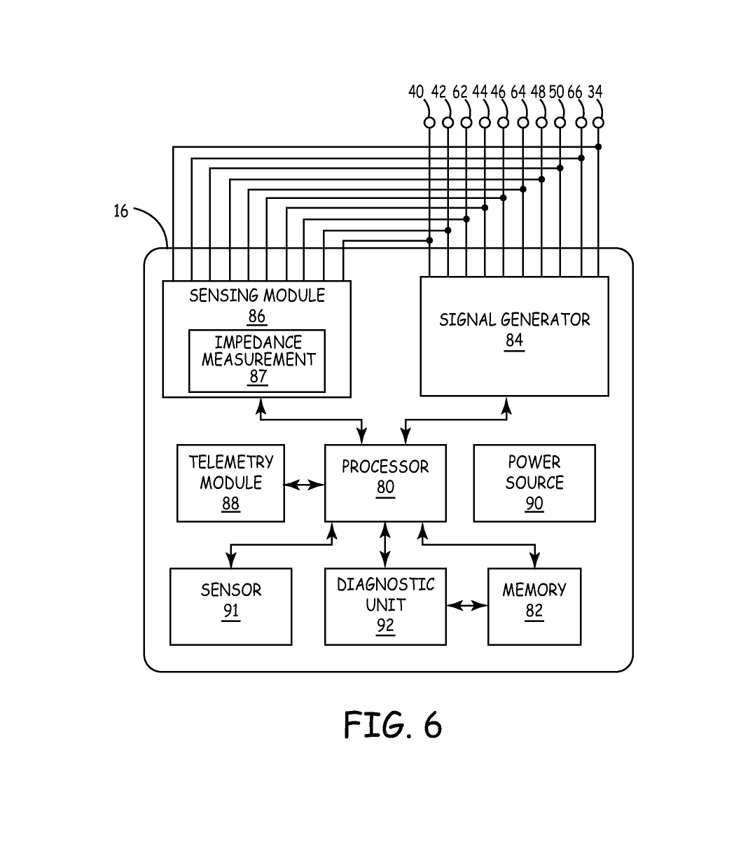

FIG. 6 is a functional block diagram of one example of IMD 16, which includes a processor 80, memory 82, signal generator 84, electrical sensing module 86, telemetry module 88, power source 90, sensor 91 and diagnostic unit 92. Processor 80 may comprise one or more processors. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. The instructions for implementing that portion of the present invention performed by the IMD are stored therein. The functions attributed to processor 80 herein may be embodied as software, firmware, hardware or any combination thereof.

Processor 80 controls signal generator 84 to deliver stimulation therapy to heart 12 based on a selected one or more of therapy programs, which may be stored in memory 82. Specifically, processor 80 may control signal generator 84 to deliver electrical pulses with the amplitudes, pulse widths, frequency, or electrode polarities specified by the selected one or more therapy programs.

Signal generator 84 is electrically coupled to electrodes 34, 40, 42, 44, 46, 48, 50, 62, 64, and 66, e.g., via conductors of the respective lead 18, 20, 22, or, in the case of housing electrode 34, via an electrical conductor disposed within housing 60 of IMD 16. A switch matrix may also be provided to connect signal generator 84 to one or more of electrodes 34, 40, 42, 44, 46, 48, 50, 62, 64, and 66.

Signal generator 84 is configured to generate and deliver electrical stimulation therapy to heart 12. For example, signal generator 84 may deliver defibrillation shocks to heart 12 via at least two of electrodes 34, 62, 64, 66. Signal generator 84 may also deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 84 delivers pacing, cardioversion, or defibrillation stimulation in the form of electrical pulses. In other examples, signal generator 84 may deliver one or more of these types of stimulation in the form of other signals, such as sine waves, square waves, or other substantially continuous time signals.

Signal generator 84 may include a switch module, and processor 80 may use the switch module to select, e.g., via a data/address bus, which of the available electrodes are used to deliver defibrillation pulses or pacing pulses. The switch module may include a switch array, switch matrix, multiplexer, transistor array, microelectromechanical switches, or any other type of switching device suitable to selectively couple stimulation energy to selected electrodes.

Electrical sensing module 86 monitors signals from at least one of electrodes 34, 40, 42, 44, 46, 48, 50, 62, 64 or 66 in order to monitor electrical activity of heart 12. Sensing module 86 may also include a switch module to select which of the available electrodes are used to sense the heart activity. In some examples, processor 80 may select the electrodes that function as sense electrodes via the switch module within sensing module 86, e.g., by providing signals via a data/address bus. In some examples, sensing module 86 includes one or more sensing channels, each of which may comprise an amplifier. In response to the signals from processor 80, the switch module within sensing module 86 may couple the outputs from the selected electrodes to one of the sensing channels.

In some examples, one channel of sensing module 86 may include an R-wave amplifier that receives signals from electrodes 40 and 42, which are used for pacing and sensing in right ventricle 28 of heart 12. Another channel may include another R-wave amplifier that receives signals from electrodes 44 and 46, which are used for pacing and sensing proximate to left ventricle 32 of heart 12. In addition, in some examples, one channel of sensing module 86 may include a P-wave amplifier that receives signals from electrodes 48 and 50, which are used for pacing and sensing in right atrium 26 of heart 12. Furthermore, in some examples, one or more of the sensing channels of sensing module 84 may be selectively coupled to housing electrode 34, or elongated electrodes 62, 64, or 66, with or instead of one or more of electrodes 40, 42, 44, 46, 48 or 50, e.g., for unipolar sensing of R-waves or P-waves in any of chambers 26, 28, 36, or 32 of heart 12.

Signals from the selected sensing electrodes that are selected for coupling to this wide-band amplifier may be provided to a multiplexer, and thereafter converted to multi-bit digital signals by an analog-to-digital converter for storage in memory 82 as an electrogram (EGM). In some examples, the storage of such EGMs in memory 82 may be under the control of a direct memory access circuit. Processor 80 may employ digital signal analysis techniques to characterize the digitized signals stored in memory 82 to detect and classify the patient's heart rhythm from the electrical signals. Processor 80 may detect and classify the patient's heart rhythm by employing any of the numerous signal processing methodologies known in the art.

IMD 16 is configured to generate and deliver pacing pulses to heart 12, processor 80 may include pacer timing and control module, which may be embodied as hardware, firmware, software, or any combination thereof. The pacer timing and control module may comprise a dedicated hardware circuit, such as an ASIC, separate from other processor 80 components, such as a microprocessor, or a software module executed by a component of processor 80, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes Intervals defined by the pacer timing and control module within processor 80 may include atrial and ventricular pacing escape intervals, refractory periods during which sensed P-waves and R-waves are ineffective to restart timing of the escape intervals, the pulse widths of the pacing pulses, A-V intervals, and V-V intervals for cardiac resynchronization therapy (CRT). As another example, the pacer timing and control module may define a blanking period, and provide signals sensing module 86 to blank one or more channels, e.g., amplifiers, for a period during and after delivery of electrical stimulation to heart 12. As another example, the pacer timing and control module may control intervals for delivery of refractory period stimulation or cardiac potentiation therapy. The durations of these intervals may be determined by processor 80 in response to stored data in memory 82. The pacer timing and control module of processor 80 may also determine the amplitude of the cardiac pacing pulses. During pacing, escape interval counters within the pacer timing/control module of processor 80 may be reset upon sensing of R-waves and P-waves.

Stimulation generator 84 may include pacer output circuits that are coupled, e.g., selectively by a switching module, to any combination of electrodes 34, 40, 42, 44, 46, 48, 50, 62, or 66 appropriate for delivery of a bipolar or unipolar pacing pulse to one of the chambers of heart 12. Processor 80 may reset the escape interval counters upon the generation of pacing pulses by stimulation generator 84, and thereby control the basic timing of cardiac pacing functions, including anti-tachyarrhythmia pacing (ATP).

The values of the counts present in the escape interval counters when reset by sensed R-waves and P-waves may be used by processor 80 to measure the durations of R-R intervals, P-P intervals, PR intervals and R-P intervals, which are measurements that may be stored in memory 82. Processor 80 may use the count in the interval counters to detect an arrhythmia event, such as an atrial or ventricular fibrillation or tachycardia.

In some examples, processor 80 may operate as an interrupt driven device, and is responsive to interrupts from pacer timing and control module, where the interrupts may correspond to the occurrences of sensed P-waves and R-waves and the generation of cardiac pacing pulses. Any necessary mathematical calculations to be performed by processor 80 and any updating of the values or intervals controlled by the pacer timing and control module of processor 80 may take place following such interrupts. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

In the event that processor 80 detects an atrial or ventricular tachyarrhythmia based on signals from sensing module 86, and an anti-tachyarrhythmia pacing regimen is desired, timing intervals for controlling the generation of anti-tachyarrhythmia pacing therapies by signal generator 84 may be loaded by processor 80 into the pacer timing and control module to control the operation of the escape interval counters therein and to define refractory periods during which detection of R-waves and P-waves is ineffective to restart the escape interval counters.

If IMD 16 is configured to generate and deliver defibrillation pulses to heart 12, signal generator 84 may include a high voltage charge circuit and a high voltage output circuit. If IMD 16 is configured to generate and deliver pacing pulses to heart 12, signal generator 84 may include a low voltage charge circuit and a low voltage output circuit. In the event that generation of a cardioversion or defibrillation pulse is required, processor 80 may employ the escape interval counter to control timing of such cardioversion and defibrillation pulses, as well as associated refractory periods. In response to the detection of atrial or ventricular fibrillation or tachyarrhythmia requiring a cardioversion pulse, processor 80 may activate a cardioversion/defibrillation control module, which may, like pacer timing and control module, be a hardware component of processor 80 and/or a firmware or software module executed by one or more hardware components of processor 80. The cardioversion/defibrillation control module may initiate charging of the high voltage capacitors of the high voltage charge circuit of signal generator 84 under control of a high voltage charging control line.

Processor 80 may monitor the voltage on the high voltage capacitor may be monitored, e.g., via a voltage charging and potential (VCAP) line. In response to the voltage on the high voltage capacitor reaching a predetermined value set by processor 80, processor 80 may generate a logic signal that terminates charging. Thereafter, timing of the delivery of the defibrillation or cardioversion pulse by signal generator 84 is controlled by the cardioversion/defibrillation control module of processor 80. Following delivery of the fibrillation or tachycardia therapy, processor 80 may return signal generator 84 to a cardiac pacing function and await the next successive interrupt due to pacing or the occurrence of a sensed atrial or ventricular depolarization.

Telemetry module 88 includes any suitable hardware, firmware, software or any combination thereof for communicating with another device, such as programmer 24 (FIG. 4). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

As illustrated in FIG. 6, sensing module 86 may include an impedance measurement module 87. Processor 80 may control impedance measurement module 87 to periodically measure an electrical parameter to determine impedance, such as intrathoracic impedance. For an intrathoracic impedance measurement, processor 80 may control stimulation generator 84 to deliver an electrical signal between selected electrodes and impedance measurement module 87 to measure a current or voltage amplitude of the signal. Processor 80 may select any combination of electrodes 34, 40, 42, 44, 46, 48, 50, 62, 64, and 66, e.g., by using switch modules in signal generator 84 and sensing module 86.

Impedance measurement module 87 includes sample and hold circuitry or other suitable circuitry for measuring resulting current and/or voltage amplitudes. Processor 80 determines an impedance value from the amplitude value(s) received from impedance measurement module 87. In some examples, processor 80 may perform an impedance measurement by causing signal generator 84 to deliver a voltage pulse between two electrodes and examining resulting current amplitude value measured by impedance measurement module 87. In these examples, signal generator 84 delivers signals that do not necessarily deliver stimulation therapy to heart 12, due to, for example, the amplitudes of such signals and/or the timing of delivery of such signals. For example, these signals may comprise sub-threshold amplitude signals that may not stimulate heart 12. In some cases, these signals may be delivered during a refractory period, in which case they also may not stimulate heart 12.

In other examples, processor 80 may perform an impedance measurement by causing signal generator 84 to deliver a current pulse across two selected electrodes. Impedance measurement module 87 holds a measured voltage amplitude value. Processor 80 determines an impedance value based upon the amplitude of the current pulse and the amplitude of the resulting voltage that is measured by impedance measurement module 87. IMD 16 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue.

In certain cases, IMD 16 may measure intrathoracic impedance values that include both a resistive and a reactive (i.e., phase) component. In such cases, IMD 16 may measure impedance during delivery of a sinusoidal or other time varying signal by signal generator 84, for example. Thus, as used herein, the term “impedance” is used in a broad sense to indicate any collected, measured, and/or calculated value that may include one or both of resistive and reactive components.

In the illustrated example shown in FIG. 6, IMD 16 includes diagnostic unit 92. Diagnostic unit 92 may provide provides the heart failure analysis capabilities as described in the above cited Sarkar, et al. application to provide a patient alert. Although diagnostic unit 92 is described as performing the various monitoring and detecting techniques proscribed to IMD 16, it should be understood that these techniques may also be performed by processor 80, e.g., that diagnostic unit 92 may be a functional module provided or executed by processor 80. Accordingly, although processor 80 and diagnostic unit 92 are illustrated as separate modules in FIG. 6, processor 80 and diagnostic unit 92 may be incorporated in a single processing unit or equivalent circuitry. Further, in some embodiments, processor 80 and diagnostic unit 92 may embody the heart failure risk assessment methodology of the present invention, operating under control of instructions stored in memory 82. However, in the disclosed embodiment, the risk score, as noted above, is derived by the server 314 (FIG. 4).

In operation, diagnostic unit 92 monitors a primary diagnostic parameter and one or more secondary diagnostic parameters according to the present invention as discussed below in more detail. In the example illustrated in FIG. 6, diagnostic unit 92 may receive signals or indications from processor 80, sensing module 86 or other sensors 91 to monitor the primary and secondary diagnostic parameters. Thus, IMD 16 may be configured to monitor physiological parameters that are capable of being sensed using any combination of electrodes 34, 40, 42, 44, 46, 48, 50, 62, 64 and 66. For example, IMD 16 may be configured to monitor intrathoracic impedance and/or electrical activity of heart 12, using any combination of electrodes 34, 40, 42, 44, 46, 48, 50, 62, 64 and 66.

IMD 16 may also be configured, in various examples, to monitor other diagnostic parameters. In some examples, IMD 16 may be configured to include other types of sensors, such as sensor 91 illustrated in FIG. 3, suitable for monitoring other primary and secondary diagnostic parameters, such as one or more pressure sensors for monitoring a cardiovascular pressure in patient 14, one or more accelerometers for monitoring the activity level of patient 14, one or more pressure sensors for monitoring the heart rate variability and night heart rate of patient 14, and/or one or more pressure sensors for monitoring the respiratory rate, depth or pattern of patient 14. In such embodiments, pressure sensors may be carried by leads 18, 20, or 22 or by one or more additional leads coupled to IMD 16. In embodiments in which IMD 16 monitors the activity level of patient 14, one or more accelerometers may be contained within or positioned on the housing of IMD 16, may be carried by one or more of leads 18, 20, and 22 or one or more additional leads, or may be a remote sensor in communication with IMD 16. In addition to fluid accumulation as a primary diagnostic parameter, in some examples, diagnostic unit 92 may monitor respiratory rate, depth or pattern of patient 14 as a secondary diagnostic parameter based on the intrathoracic impedance determined based on signals received from impedance measurement module 87. In some examples, IMD 16 may include sensors, such as chemical, pressure or fluid sensors, for monitoring renal function. Furthermore, in some examples, diagnostic unit 92 may receive signals or information from external sources, such as programmer 24 or an external sensor, such as a scale, and monitor such information or signals as secondary diagnostic parameters. Additionally, diagnostic unit 92 may receive information from processor 80, or may maintain information in memory 82, indicating percentage of CRT pacing as a secondary diagnostic parameter. Diagnostic unit 92 or processor 80 may determine whether or not CRT pacing is delivered based on information from processor 80 of a pacer timing and control module thereof.

If diagnostic unit 92 detects a risk of worsening heart failure of patient 14, using the methodology described in the above cited Sarkar application, it may optionally provide an alert to patient 14. Diagnostic unit 92 may include or be coupled to an alert module (not shown) that provides, as examples, an audible or tactile alert to patient 14 of the worsening heart failure. In some examples, diagnostic unit 92 additionally or alternatively provide an indication of worsening heart failure to programmer 24 or another device via telemetry module 88 and/or network, which may provide an alert to a user, such as patient 14 or a clinician.

In some embodiments of the invention, the diagnostic unit may also determine whether changes in therapy delivered by the device are necessary due to changes in the monitored fluid content using the Opti-vol index described above. Methodologies for control of therapies delivered based upon fluid measurements may be as described in U.S. Pat. No. 7,127,290, issued to Girouard, et al. on Oct. 24, 2006 or in U.S. Pat. No. 7, 659,899, issued to Sachanandini, et al on Dec. 8, 2009.

The various components of IMD 16 are coupled to power source 90, which may include a rechargeable or non-rechargeable battery. A non-rechargeable battery may be capable of holding a charge for several years, while a rechargeable battery may be inductively charged from an external device, e.g., on a daily or weekly basis.

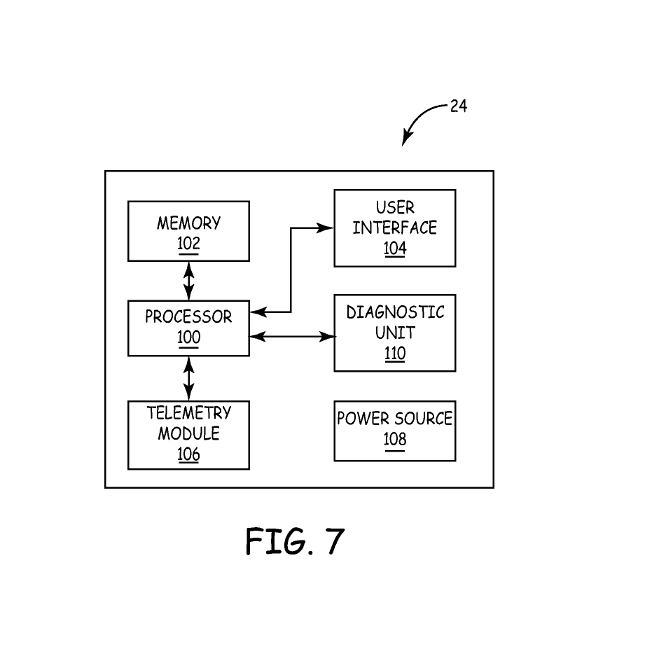

FIG. 7 is block diagram of an example programmer 24. As shown in FIG. 7, programmer 24 includes processor 100, memory 102, user interface 104, telemetry module 106, and power source 108. In some examples, programmer 24, as illustrated in FIG. 4, includes a diagnostic unit 110. Programmer 24 may be a dedicated hardware device with dedicated software for programming of IMD 16.

Alternatively, programmer 24 may be an off-the-shelf computing device running an application that enables programmer 24 to program IMD 16. A user may use programmer 24 to select worsening heart failure detection algorithms, e.g., select diagnostic parameters from a list of possible diagnostic parameters. A user may also use programmer 24 to configure other sensing or any therapy provided by IMD 16. The clinician may interact with programmer 24 via user interface 104, which may include display to present graphical user interface to a user, and a keypad or another mechanism for receiving input from a user.

Although illustrated and described in the context of examples in which programmer 24 is able to program the functionality of IMD 16, in other examples a device capable of communicating with IMD 16 and providing functionality attributed to programmer 24 herein need not be capable of programming the functionality of the IMD. For example, an external home or patient monitor may communicate with IMD 16 for any of the purposes described herein, but need not independently be capable of programming the functionality of the IMD. Such as a device may be capable of communicating with other computing devices via a network, as discussed in greater detail below.

Processor 100 can take the form one or more microprocessors, DSPs, ASICs, FPGAs, programmable logic circuitry, or the like, and the functions attributed to processor 100 herein may be embodied as hardware, firmware, software or any combination thereof. Diagnostic unit 110, although illustrated as a separate module in FIG. 6, may be incorporated in a single processing unit with processor 100 or functional module executed or provided by processor 100.

Memory 102 may store instructions that cause processor 100 and/or diagnostic unit 110 to provide the functionality ascribed to programmer 24 herein, and information used by processor 100 and/or diagnostic unit 110 to provide the functionality ascribed to programmer 24 herein. Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before programmer 24 is used to program therapy for another patient. Memory 102 may also store information that controls operation of IMD 16, such as therapy delivery values.

A user, such as a clinician, technician, or patient 14, may interact with programmer 24 via user interface 104. User interface 106 may include display to present graphical user interface to a user, and a keypad or another mechanism for receiving input from a user. In some examples, user interface 106 may include a touch screen display. In preferred embodiments, as discussed below, programmer 24 may be employed to display the heart failure risk score and associated information provided by the present invention. In some embodiments, the heart failure risk assessment methodology of the present invention may be embodied within the processor 100 and/or diagnostic unit 110 under control of stored instructions in memory 102, as an alternative to its embodiment within the server as discussed above.

Programmer 24 may communicate wirelessly with IMD 16, such as using RF communication or proximal inductive interaction. This wireless communication is possible through the use of telemetry module 106, which may be coupled to an internal antenna or an external antenna. An external antenna that is coupled to programmer 24 may correspond to the programming head that may be placed over heart 12, as described above with reference to FIG. 1. Telemetry module 106 may be similar to telemetry module 88 of IMD 16 (FIG. 3).

Programmer 24 may also be configured to communicate with another computing device via wireless communication techniques, or direct communication through a wired, e.g., network, connection. Examples of local wireless communication techniques that may be employed to facilitate communication between programmer 24 and another computing device include RF communication based on the 802.11 or Bluetooth specification sets, infrared communication, e.g., based on the IrDA standard.

Power source 108 delivers operating power to the components of programmer 24. Power source 108 may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within programmer 24. In other embodiments, traditional batteries (e.g., nickel cadmium or lithium ion batteries) may be used. In addition, programmer 24 may be directly coupled to an alternating current outlet to power programmer 24. Power source 108 may include circuitry to monitor power remaining within a battery. In this manner, user interface 104 may provide a current battery level indicator or low battery level indicator when the battery needs to be replaced or recharged. In some cases, power source 108 may be capable of estimating the remaining time of operation using the current battery.

In some examples, IMD 16 may detect worsening heart failure using any of the techniques described herein, and provide an indication of worsening heart failure to programmer 24. In such examples, programmer 24 need not include diagnostic module 110. Processor 100 may control user interface 106 to provide an alert of worsening heart failure of patient 14 to the patient, a clinician, or other users. In some examples, processor 100 may provide an alert of worsening heart failure of patient 14 to one or more computing devices via a network. A user may use programmer 24 to retrieve and/or view data regarding primary and secondary diagnostic parameters.

In some examples, programmer 24 includes diagnostic module 110 that receives diagnostic data from IMD 16, or other implanted or external sensors or devices, i.e., data regarding the primary and secondary diagnostic parameters, and processes the received data to detect worsening heart failure in patient 14. In this manner, diagnostic unit 110 may perform substantially the same functionality as described with respect to diagnostic unit 92 in FIG. 3. IMD 16 may not need to include diagnostic unit 92 in examples in which programmer 24 includes diagnostic unit 110. Diagnostic unit 110 may include an alert module that provides an alert to patient 14 or a clinician via user interface 104 when worsening heart failure is detected in patient 14, and/or provides a notification to one or more computing devices via a network.

Alerts provided via user interface 104 may include a silent, audible, visual, or tactile alert. For example, user interface 104 may emit a beeping sound, display a text prompt, cause various buttons or screens to flash, or vibrate to alert patient 14 or another user that a heart failure decompensation event may be likely to occur. Patient 14 may then seek medical attention, e.g., check in to a hospital or clinic, to receive appropriate treatment, or the other user may instruct patient 14 to do so.

Although illustrated and described in the context of examples in which programmer 24 is able to program the functionality of IMD 16, in other examples a device capable of communicating with IMD 16 and providing functionality attributed to programmer 24 herein need not be capable of programming the functionality of the IMD. For example, an external home or patient monitor may communicate with IMD 16 for any of the purposes described herein, but need not independently be capable of programming the functionality of the IMD. Such as a device may be capable of communicating with other computing devices via a network, as discussed in greater detail below.

### The Bavesian Network Based Approach.

A plethora of common statistical approaches could be applied in order to develop an integrated heart failure diagnostic. However, the Bayesian approach has several potential advantages that make it an attractive option for such applications.

The standard Frequentist approach to statistics defines the uncertainty of an event (or random variable A) in terms of the probability of the event happening (or Pr(A)). For example, in order to evaluate the probability of HF decompensation (HFD) in a patient with CRT-D devices in 12 months, one would count the number of patients having HFD in a year then divide by the total number of patients to determine the Pr(HFD). In the Bayesian approach, the Frequentist approach is augmented to include other available information or evidence to re-estimate Pr(HFD) in the present environment (i.e. in the context of the evidence that is available).

For example, assume one wishes to know the probability of HFD given that the OptiVol fluid index has crossed threshold (Pr(HFD|OptiVol). The OptiVol fluid Index crossing threshold does not necessarily imply an impending HFD. Thus, we employ uncertain reasoning, or probabilities, and further apply the Bayesian approach to quantify the probability given the existing current diagnostic evidence.

Using Bayes rule:

Pr(HFD|OptiVol)=Pr(OptiVol|HFD)*Pr(HFD)/Pr(OptiVol)
Where,
   Pr(HFD|OptiVol) is the posteriori belief (or what we want to find)
   Pr(OptiVol|HFD) is the likelihood (which we know from prior data or expert knowledge)
   Pr(HFD) is the prior belief (which is the prior belief given no evidence)
   Pr(OptiVol) is the normalization factor in this case (can be computed from prior data)

Thus we can estimate the posterior belief of HFD given OptiVol evidence using the likelihood ratio of OptiVol evidence being present before HFD and the prior belief of HFD normalized by the probability of the OptiVol evidence. One aesthetic advantage of this approach is that it is analogous to how the human brain “thinks”; the Bayesian approach just formalizes that approach in a mathematical framework.

When there are multiple variables involved, Bayes' rule may be expanded. The posterior probability computations will then involve computing joint probability distributions and defining multiple combinations of conditional probabilities. One limitation to more broad application of Bayesian probability is that computation of the joint and conditional probability distributions become prohibitive when the number of variables increases. Bayesian Belief network theory addresses that problem by assigning explicit relationships between variables, thereby making the computation more feasible. With explicit defined relationships between variables, only the conditional and joint probabilities for the associated variables need to be defined. Thus Bayesian Belief networks provide a framework for assumptions regarding the explicit relationship between variables to make computation more feasible.

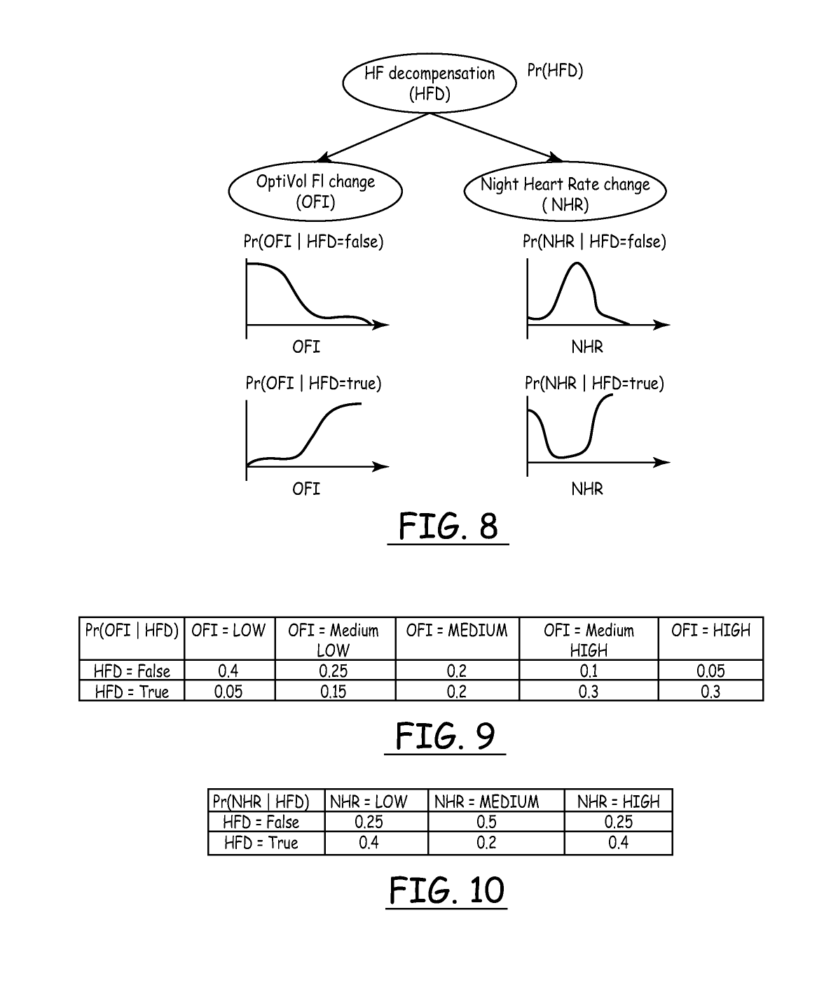

FIG. 8 illustrates is an example of a simple Bayesian Belief Network. The network consists of three variables including HF decompensation (HFD), OptiVol Fluid Index (OFI), and Night Heart Rate (NHR). The explicit causal relationship that is assumed in this network is that HFD may cause both OFI changes and NHR changes. The goal of this network may be to find Pr(HFD|OFI, NHR) i.e. what is the probability of HFD given evidence about OFI and/or NHR. In addition, it is desirable to find Pr(OFI|NHR) or Pr(NHR|OFI) i.e. what is the probability of OFI if only NHR evidence is available or vice versa.

To obtain Pr(HFD|OFI, NHR), the likelihood and the prior probability needs to be defined for the network. In this example, Pr(OFI|HFD) and Pr(NHR|HFD) are the likelihood estimates, and Pr(HFD) being the a priori probability of HFD, which needs to be defined based on prior data or expert knowledge. The likelihood estimates can be defined in form of continuous probability distributions as shown in FIG. 8. However, for easier computation, the likelihood estimates can be defined as conditional probability tables. Each of the variables OFI and NHR can be converted to discrete states. For example, OFI can be discretized to five ranges of values [LOW, Medium LOW, MEDIUM, Medium HIGH, HIGH] and the conditional probability table simplifies to the table illustrated in FIG. 9.

Each value in the table of FIG. 9 represents the probability the OFI is in one of the five states given HFD is known to be true or false. Note, that by rules of probability, the sum for each row should be 1. Similarly, NHR can be discretized to 3 ranges of values [LOW, MEDIUM, HIGH] and to the simplified conditional probability table illustrated in FIG. 10.

The basic three variable networks can be expanded to larger numbers of variables. So, for the integrated diagnostics problem, we want to know Pr(HFD|Optivol, NHR, HRV, Activity, AF, VT/VF, % VP, Weight, BP, prior HF events, symptoms). Therefore, we expand the Bayesian Belief network (BBN) to a 12 variable network. The basic assumptions made to define the network are HFD may cause all the other variables to change, HFD is causally linked to all the other variables. All the other variables are conditionally dependent on HFD, i.e. if there is evidence of HFD (either TRUE or FALSE), then the other variables are associated with each other, otherwise they are independent of each other, i.e. the other variables are not linked to each other but are only linked through HFD.

Any Bayesian network problem may have multiple realizations of a BBN depending on the prior assumptions. For example, a high NHR may cause AT/AF or VT/VF independent of the HF condition, but, for this specific model realization, it is assumed that this cannot be the case. However, this basic BBN can be upgraded to incorporate those dependencies.

Similar to the three variable examples, for this BBN network the conditional probabilities for the additional variables need to be specified. Additionally we need to specify Pr(Activity|HFD), Pr(HRV|HFD), etc. All conditional probabilities can be derived using either prior data or expert knowledge. Thus, when we obtain evidence on the state of the different variables, that is input in the network and the BBN applies that information through the entire network. For example, if we only have OptiVol evidence, this updates the Pr(HFD) and since all the other variables are linked to HFD, the probability for those other variables are also updated if the evidence regarding them are not present. Similarly, if weight information is not available because the patient did not weigh themselves, then the information that OptiVol is HIGH also increases the probability that weight is also HIGH assuming that the conditional probability tables defines that for both OptiVol and weight being HIGH increases the probability of HFD.

### Defining the Prior Probability

Multiple existing clinical databases were used to determine the baseline variables that are associated with the HFD event. All available baseline variables were used in a univariate and multivariate logistic regression. The baseline variables collected at the beginning of the study were used as the independent variables, and all HFD events in the first 6 months were used as the dependent variables in the multivariate logistic regression model. The multivariate logistic regression model fits the data and identifies the independent predictors of HFD events. Some known risk factors are always included in the model irrespective of statistical significance.

The parameters that were used in the model included Age, Sex, Weight, Height, body mass index (BMI), NYHA, ejection fraction (EF), QRS duration, Ischemic Etiology, CRT/ICD device, Hypertension, atrial arrhythmias (AF), Diabetes, coronary artery disease (CAD), MI, LBBB, RBBB, bradycardia, sinus node disease (SND). Notable exclusions from the model are baseline medications and bio-markers which are known predictors of HFD. These parameters were excluded because of unavailability of data.

The logistic regression model generates the list of independent predictors and a baseline probability table. The probability table lists all possible combination of the selected baseline parameters as well as the associated probability of a HFD event in the first six months. The generated baseline probability table from the CONNECT study is used as the prior probability Pr(HFD) for evaluating all the studies. Thus, for each patient, the states for each of the selected baseline parameters are determined from Case Report Forms. The prior probability Pr(HFD) for the patient is determined by table look up from the baseline probability table, i.e., a given set of states for all the selected baseline parameters will correspond to one particular row of the baseline probability tables from which the Pr(HFD) is determined.

Defining the Likelihood tables

The likelihood tables are defined based on the data from the development set. The development set comprised of the following: 1. All patients from the OFISSER study; 2. Patients from the Italian Clinical Services with OptiVol feature in “Observation Only” mode; 3. Patients from the CONNECT study who had a CRT device

Each set of individual patient data is divided into 30 day periods beginning 60 days after implant. For each 30-day period, all the diagnostic variable criteria were computed. Diagnostic variable criteria map the raw diagnostic variable data into discrete risk or criteria states. One criterion state is assigned for each variable with precedence given to a “risky” state. For example, if for any day during the 30 day period, OptiVol met the condition for criteria state 4 then the criteria state of 4 is assigned for the OptiVol diagnostic for that 30-day period irrespective of whether any other criteria state was met during that 30-day period. For each 30-day period it was also evaluated whether an HFD event occurred within the next 30-day period. Thus, the likelihood ratios could be computed as Pr(Diagnostic=Criteria State|HFD=False in the next 30 day period)=Number of 30-day period with “Diagnostic=Criteria State” and the following 30-day period without a HFD event/Total number of 30-day periods without a HFD event.

Similarly, Pr(Diagnostic=Criteria State|HFD=True in the next 30 day period)=Number of 30-day period with “Diagnostic=Criteria State” and the following 30-day period with a HFD event/Total number of 30-day periods with a HFD event. Therefore, the conditional probability table or the likelihood tables can be computed for each criteria state for each diagnostic variable.

### Generating the BBN Probability Tables

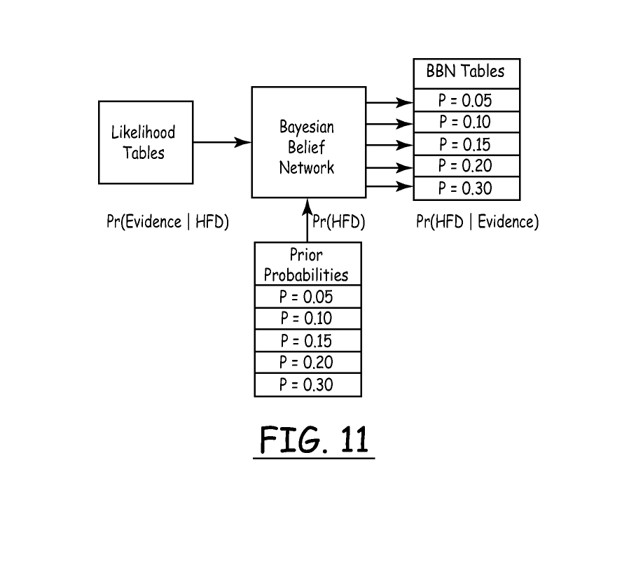

The likelihood tables defined for each criteria state for each diagnostic variable are then provided as input to the Bayesian Belief Network model implemented in the BBN toolbox in Matlab. The BBN model then can provide the posterior probability of Pr(HFD|Diagnostic variable=Criteria State). This posterior probability is then tabulated for all possible combinations of Diagnostic variable and Criteria State to create the BBN Probability tables. Once the criteria state for each diagnostic variable is assigned, the posterior probability is determined from the BBN probability tables. The scheme for generating the BBN probability tables is shown in FIG. 11.

In summary, 1. Prior probabilities are generated from the baseline and clinical events data; 2. The likelihood tables are generated from data as explained previously; and 3. The BBN tables are generated using the BBN toolbox in Matlab.

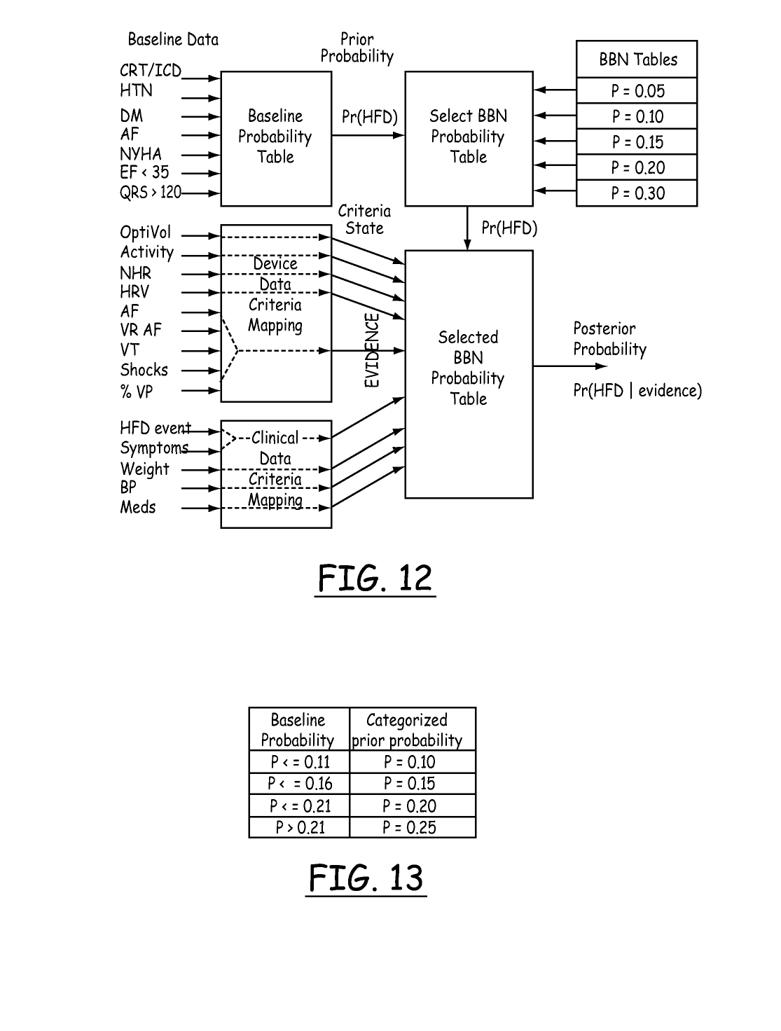

The algorithm implementation comprises the following steps as outlined in the schematic shown in FIG. 12: 1. Generating prior probability estimate and selecting corresponding BBN table; 2. Criteria State Mapping; and 3. Generating posterior probability.

### Generating Prior Probability Estimate and Selecting Corresponding BBN Table

The states of the baseline, or static, variables are to be obtained for each patient at implant. The baseline information is used to look up the prior probability from the baseline probability table. The prior probability is categorized into four possible values (0.1, 0.15, 0.20, 0.25). This process limits the number of BBN probability tables to be used. The table of FIG. 13 describes this categorization. The corresponding BBN table is selected based on the categorized prior probability estimate.

### Criteria State Mapping

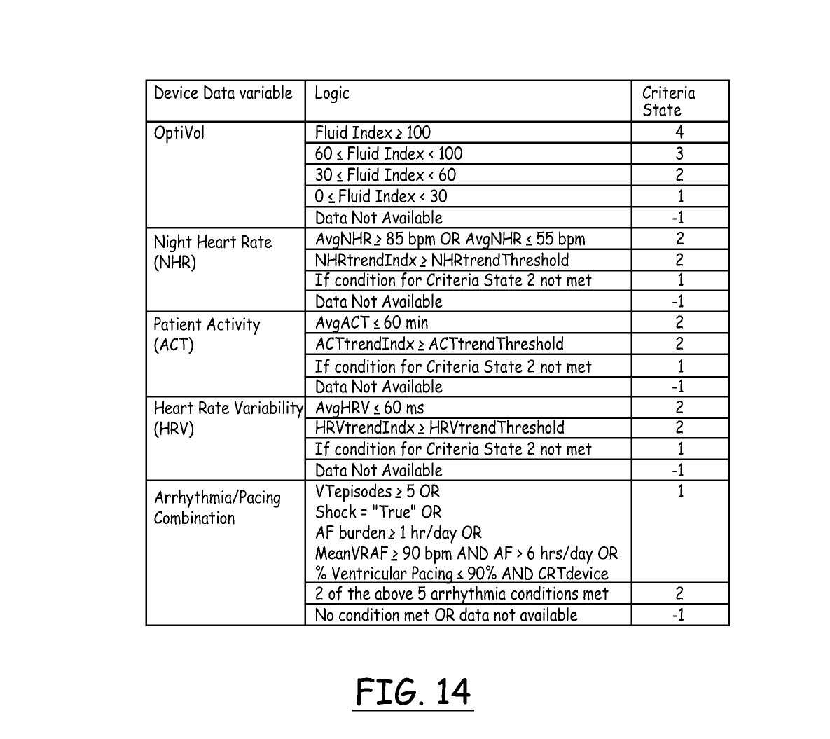

Criteria state mapping for device data collected in the long term clinical trends (cardiac compass) was performed per the logic illustrated in the Table of FIG. 14.

### Additional Computation for Activity, NHR, and HRV

The three parameters need additional computations which are similar to computation of the OptiVol Fluid Index. The purpose of these computations was to establish whether these parameters increased or decreased over a period of time. All the computation for the above three parameters are similar.

The word PARAM will be used for following description of the computations.

Average PARAM is computed every day as the average of last 7 days of PARAM values. Average PARAM can be computed only if 4 out of the last 7 days have valid measurements else it is undetermined.

Long term average PARAM for a given day is computed as the average of 4 average PARAM values of the present day, present day—7days, present day—14 days, and present day—21 days. The increase or decrease of the long term average PARAM is limited to DRIFT DOWN and DRIFT UP. Long term average PARAM can be only be computed if average PARAM can be computed for at least one day in the last 14 days else it is undetermined.

Daily Difference PARAM for a given day is the difference between the long term average PARAM and the average PARAM. If average PARAM is undetermined then daily difference PARAM is also undetermined.

Positive difference count is incremented on days long term average PARAM is=average PARAM. It is reset to 0 if daily difference PARAM changes sign and daily difference PARAM is=0. It is also reset to 0 if negative difference count reaches 4 and positive difference count is not equal to 4. If both positive and negative difference count is 4 then positive difference count is reset if daily difference PARAM is<0.

Negative difference count is incremented on days long term average PARAM is<average PARAM. It is reset to 0 if daily difference PARAM changes sign and daily difference PARAM is<0. It is also reset to 0 if positive difference count reaches 4 and negative difference count is not equal to 4. If both positive and negative difference count is 4 then negative difference count is reset if daily difference PARAM is=0.

PARAM Positive Accumulated Difference is the sum of daily difference PARAM for a period of the last positive difference count days. If positive difference count is>14 then accumulation is done only for the last 14 days. PARAM Positive Accumulated Difference has a minimum value of 0.

PARAM Negative Accumulated Difference is the sum of daily difference PARAM for a period of the last negative difference count days. If negative difference count is>14 then accumulation is done only for the last 14 days. PARAM Negative Accumulated Difference has a maximum value of 0.

PARAM Positive Accumulated Difference=PARAM Positive Threshold or PARAM Negative Accumulated Difference=PARAM Negative Threshold (depending on the parameter as listed in the table below) for setting the criteria state for the respective parameters.

PARAM Positive Threshold is equal to Long term average PARAM*Threshold Factor. PARAM Positive Threshold cannot be less that Threshold Floor or greater than Threshold Ceiling.

PARAM Negative Threshold is equal to Long term average PARAM*Threshold Factor. PARAM Positive Threshold cannot be less that Threshold Floor or greater than Threshold Ceiling.

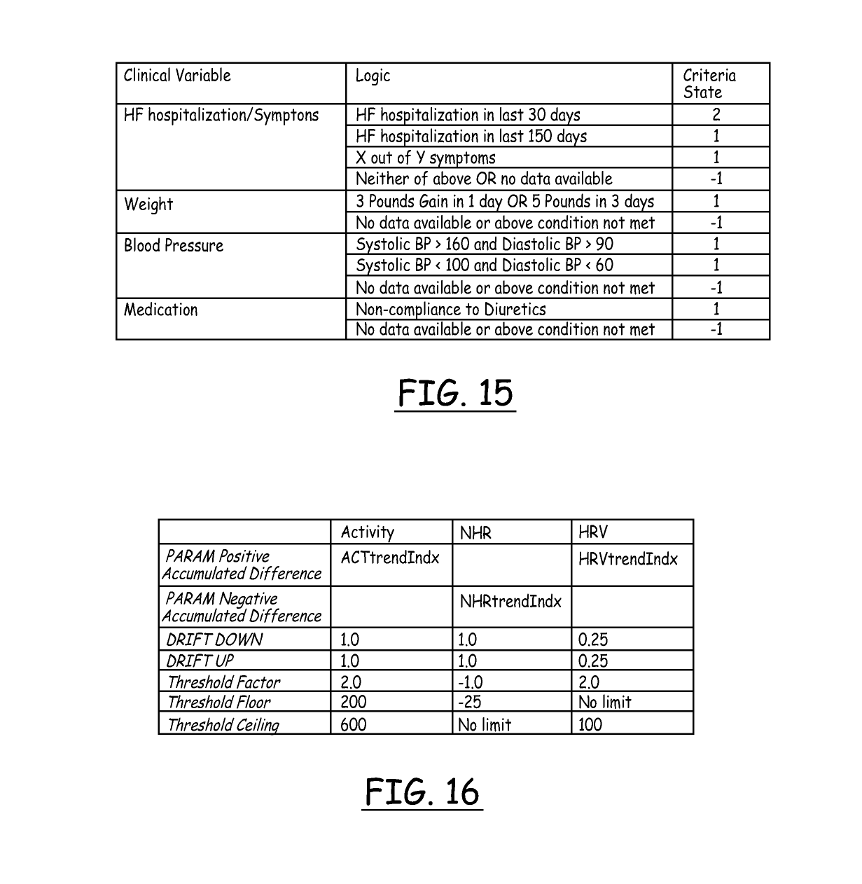

The table of FIG. 15 lists the differences between Activity, NHR, and HRV computations.

Criteria state mapping for clinical variables collected using patient management system was performed as per the logic described in the table illustrated in FIG. 16.

### Generating Posterior Probability

The criteria state information is used to look-up the posterior probability from the selected BBN table. The posterior probability is the HF risk score or the probability of a HF event given all the evidence (or criteria states) for the different diagnostic variables. At implant, the HF risk score will be same as the baseline probability which is the risk for a HF hospitalization in the next six months. Every month the HF risk score is updated based on diagnostic data from the previous month to indicate whether the imminent risk for a HF event has increased or decreased from the baseline risk in the patient. Thus, the baseline HF risk score is the overall (static) risk over a longer time frame, while the month-to-month HF risk score will be able to provide time-varying (dynamic) information regarding the time period during which the patient is more likely to have an event. FIG. 17 illustrates the variation of the resultant HF Risk Score for an exemplary patient over a 10 month period.

The HF risk score can be computed in two ways.

1. Maximum of Daily Scores: For each day, the HF risk score is calculated based on the criteria states on that day. On the follow-up day, the maximum HF risk score during the past 30 days is used as the risk score at follow-up. A high HF risk score requires multiple diagnostic criteria to be met at the same time.

2. Monthly Score: For each day only the criteria states are evaluated. On the follow-up day criteria states on the last 30 days are evaluated and the riskiest state on any given day on the last 30 days is assigned as the criteria state at follow-up. A HF risk score is then computed based on the criteria state assigned at follow-up based on the criteria state in the last 30 days. A high risk score does not need multiple diagnostic criteria to be met on the same day, but needs multiple criteria to be met in a 30 day time frame. This allows for one diagnostic criteria being a cause for another criteria to be met at a future date.

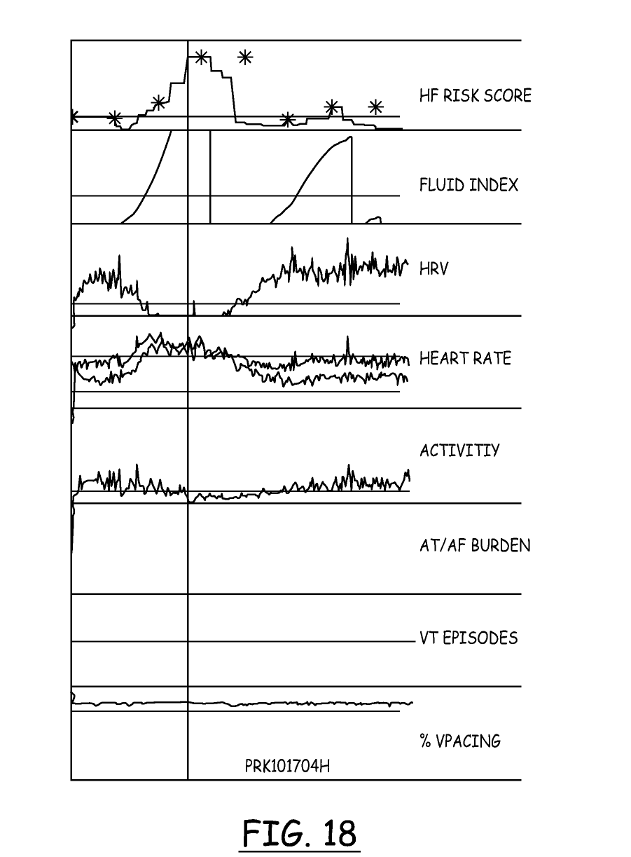

An exemplary patient case is shown in FIG. 18. The daily computed HF risk score is plotted in the top panel with the “Max of Daily” HF risk score evaluated every month being indicated by the asterisk symbol on the same panel. The ‘max of daily’ is the maximum value of the daily HF risk score in the last thirty days. This patient has two events, the first event is a more critical HF hospitalization event, and the second event is a softer HF signs and symptom event. OptiVol fluid index reaches a very high value prior to both events. However, the HF risk score value reaches a very high value prior to the HF hospitalization event only as there is more evidence from other parameters such as increasing and high NHR, decreasing and low HRV, and low activity. Thus, integrating multiple parameters provides the ability to differentiate the criticality of the event.

### Databases

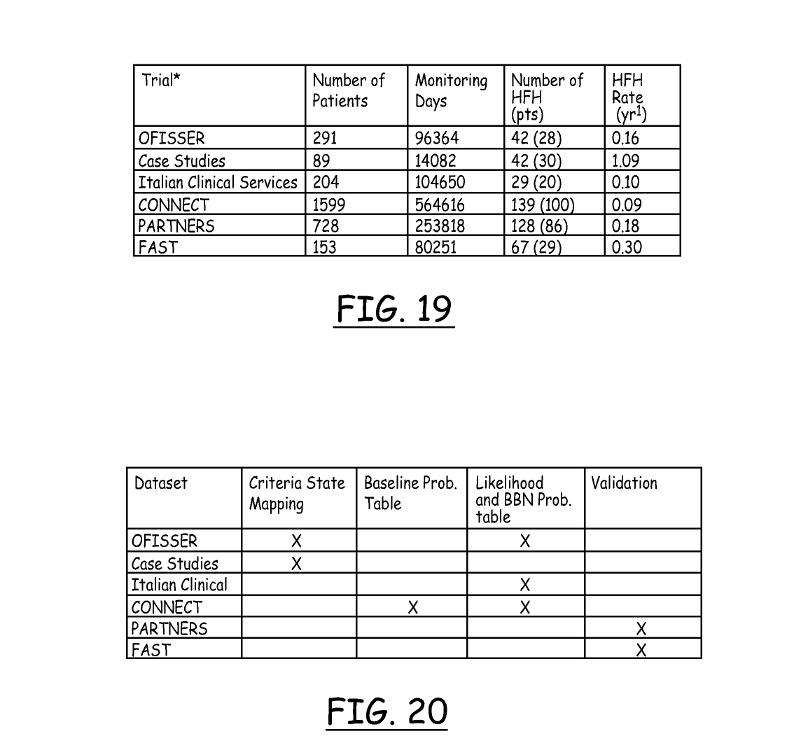

The present invention was developed and validated using datasets as illustrated in the table of FIG. 19

Each database was unique in some capacity and the characteristics are described below. The interpretation of the results and the optimization process of the algorithm was performed with due consideration to the nature of the particular dataset.

OFISSER was a retrospective registry study using CRT-D devices with the OptiVol feature. The study retrospectively reviewed patient records to identify HF clinical events and associate them with the OptiVol data. Physicians were semi-blinded to the OptiVol data as the audible alert is disabled in the US patients. The physicians had the opportunity to look at the data during the entire data collection period and change treatment plans. Clinical information, including HF hospitalization (HFH), symptoms and medications were collected. The HFH was adjudicated internally.

Case Studies: A collection of cases that were submitted to the marketing department along with clinical information. The datasets were collected in a biased manner to show the utility of the OptiVol feature. The physicians were semi-blinded to the OptiVol as the audible alert is disabled in the US patients. The physicians had the opportunity to retrospectively look at the data during follow-up and change treatment plans. A large number of HFH events in the datasets were only used to develop criteria state mapping algorithms for diagnostic parameters: activity, NHR, and HRV.

Italian Clinical Services is a service provided by the Medtronic Italy team. Physicians submit patient data to the team who manages the data and provide reports to the physicians. This dataset is also used to generate publications. Since this is not a controlled clinical study, physicians use the entire feature set in the device for routine clinical practice and hence were not blinded to the OptiVol data. In most patients the audible alert feature was ON and hence the patients heard an audible alert every day the Fluid Index was above threshold. The complete dataset is very large with a full suite of device diagnostic features available. Clinical data was collected and adjudicated internally by the Medtronic Italy team for HF hospitalizations. About 20-30% of the patients had the OptiVol patient alert disabled for long periods of time (Table 5). These patients are used for development of the methodology of the present invention.

CONNECT was a post-market study using CRT-D and ICD devices randomized with one arm having the AT/AF alerts ON and the other arm having the AT/AF alerts OFF. Physicians were semi-blinded to the OptiVol data as the audible alert is disabled in the US patients. The physicians had the opportunity to retrospectively review the data during follow-up and change treatment plans. Clinical information, including HFH, symptoms and medications were collected during the course of the study. The data collection in the study is still ongoing. The data set used to develop the present embodiment of the invention report is a freeze of the data collected prior to February of 2009. This interim dataset is very large with a full suite of device diagnostic features available. The HF hospitalizations were adjudicated internally. Only CRT-D patients were used for development of the algorithm.

PARTNERS-HF was a post-market observational study using CRT-D devices to show how the diagnostic variables are correlated with HF events. Physicians were semi-blinded to the OptiVol and diagnostics data as the audible alert is disabled in the US patients. The physicians had the opportunity to retrospectively review the data during follow-up and change treatment plans. Clinical information, including HFH, symptoms and medications were collected during the course of the study. The HF events were adjudicated by an independent adverse event adjudication committee. The full suite of device diagnostics was available in a large number of patients. The PARTNERS-HF data set is used as a validation data set.

FAST: In this study, intra-thoracic impedance diagnostic data was collected in a blinded fashion using downloaded software. Physicians were not blinded to the cardiac compass data which is a standard feature set Medtronic co0mmercially available devices. Data was collected in mostly CRT and in some dual chamber ICD devices. HFH and Worsening Heart Failure (WHF) information was collected during the study and adjudicated by an Adverse Event Adjudication Committee. The full suite of device diagnostics was available. Weight data was also available for most patients. This is the most complete dataset amongst all datasets used in this work. No lead maturation phase was included in the data, since OptiVol was downloaded into existing devices (i.e. no implant). The FAST dataset is used as a validation data set.

The table of FIG. 20 summarizes how the different studies were used for development and validation of the HF Risk Score methodology of the present invention.

### Statistical Analysis

The HF Risk Score methodology was developed for a “follow-up based” usage model. That is, the analysis methods intend to evaluate the utility of the algorithm to predict an HF event in the following month. This is based on the monthly follow-up currently encouraged by CPT codes. An HF event is defined as HF hospitalization with pulmonary congestion and/or signs and symptoms of HF. In some data sets ER visits or unscheduled clinic visits with administration of IV diuretic was also considered a HF event. Recently, reimbursement codes have been in place for a monthly follow-up based investigation of device HF diagnostics.

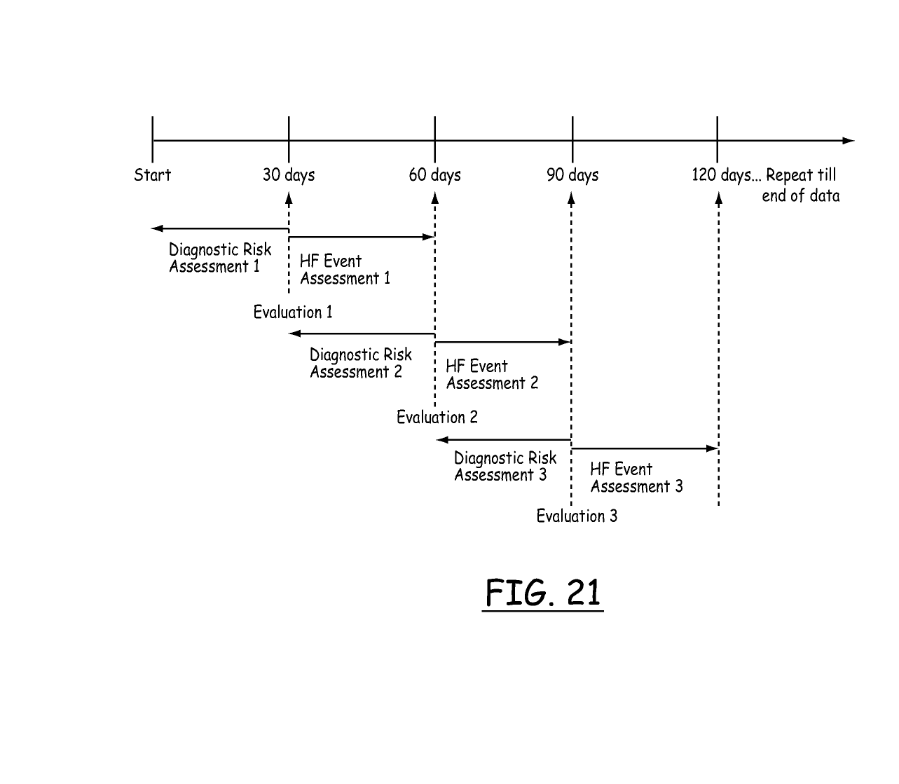

FIG. 21 illustrates the evaluation method used for the follow-up based usage model. The “Start” of data is considered to be 30 days after the first available daily OptiVol Fluid Index. For example, the “Start” day for a patient with data available from implant, will be day 64, including the 34 days prior to fluid index initialization, plus the additional 30 days. After the “Start’ day, the diagnostic variables are evaluated by the algorithm at a simulated follow-up every 30 days.

At each evaluation, the algorithm looks back at the last 30 days of data and computes two different risk scores as defined previously: 1. the Maximum of daily HF risk score and 2.the Monthly HF risk score HF clinical events are evaluated for present evaluation in the 30 days period immediately following the simulated follow-up.

Baseline probability tables were generated using a multivariate logistic regression model. The risk scores generated from the algorithm were evaluated using a Generalized Estimating Equation model (GEE). This model allows for repeated observations within a patient. Risk scores were evaluated as a continuous variable as well as by quartiles.

### Selection of Baseline Parameters

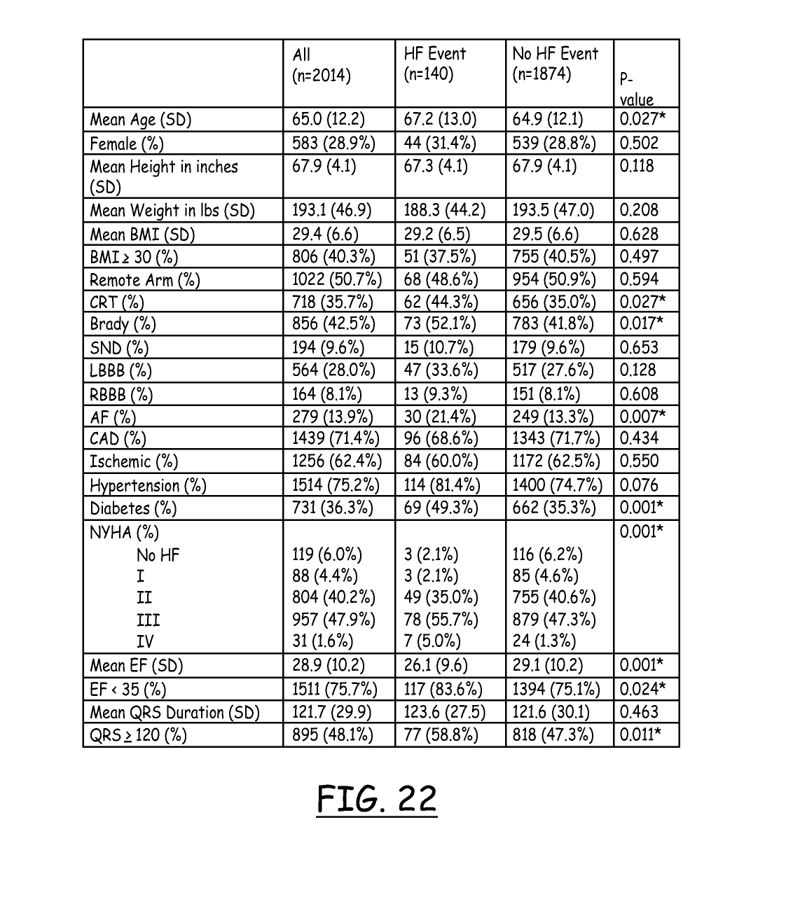

The CONNECT study data was used to evaluate the baseline probabilities and define the baseline probability tables. The study had 2014 patients with 140 patients (probability of event=0.07) having 186 HF-related events during the follow-up period that was evaluated. Considering only the first 6 months of follow-up, 84 patients had at least one HF event giving a probability of event of 0.04. Note that the data collection in the study is ongoing and these results are based on the data freeze performed on February 2009. The baseline characteristics for the different patients are shown in the table of FIG. 22. Note that patients with a HF event are more likely to be older, have a CRT device, have Bradycardia, have a history of AF, have Diabetes, have a higher NYHA class, have lower EF and EF<35%, and have QRS duration>120 ms.

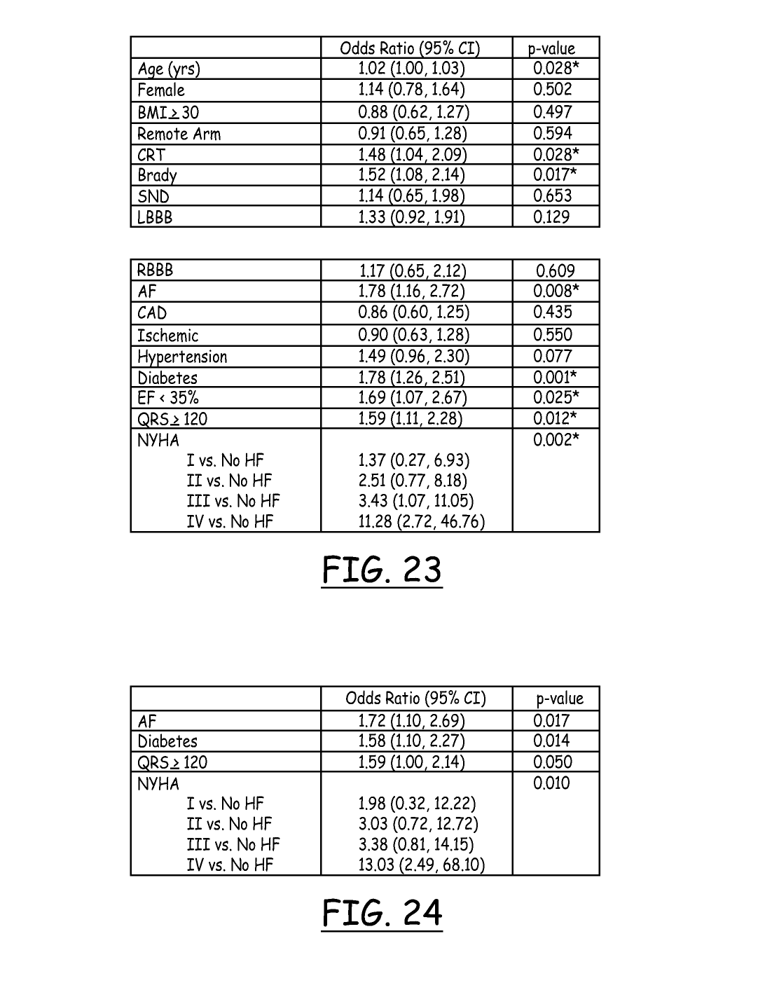

The results of univariate and multivariate logistic regression of the baseline variables against the presence or absence of a HF event are shown in the illustrated in FIGS. 23 and 24, respectively. Based on the results the following baseline parameters were chosen to be input as parameters to determine the prior probability for the algorithm: 1. Hypertension; 2. History of AF; 3. Diabetes; 4. NYHA class; 5. EF<35; 6. QRS duration=120 ms; 7. CRT/ICD device

Hypertension is historically known to be a risk factor for HF event. In the Italian data, Hypertension came out also as an independent risk factor for HF event. Further, whether the patient has a CRT/ICD device is also included such that the baseline probabilities can be computed differently for the different patient groups. It should be noted that in the CONNECT dataset for only the CRT-D patients, AF was the only significant independent risk factor and for dual chamber ICD patients. Diabetes and QRS duration=120 ms were the only independent predictors of HF events. There were several other known risk factors (such as anemia, renal dysfunction, baseline medications, and biomarkers such as BNP, and creatinine) that were excluded from this analysis due to unavailability of data.

The selected parameters were then used to generate the baseline probability table (using the same multivariate logistic regression model) based on which the prior probability is assigned at the start of HF risk score evaluation.

### Generation of Likelihood Tables

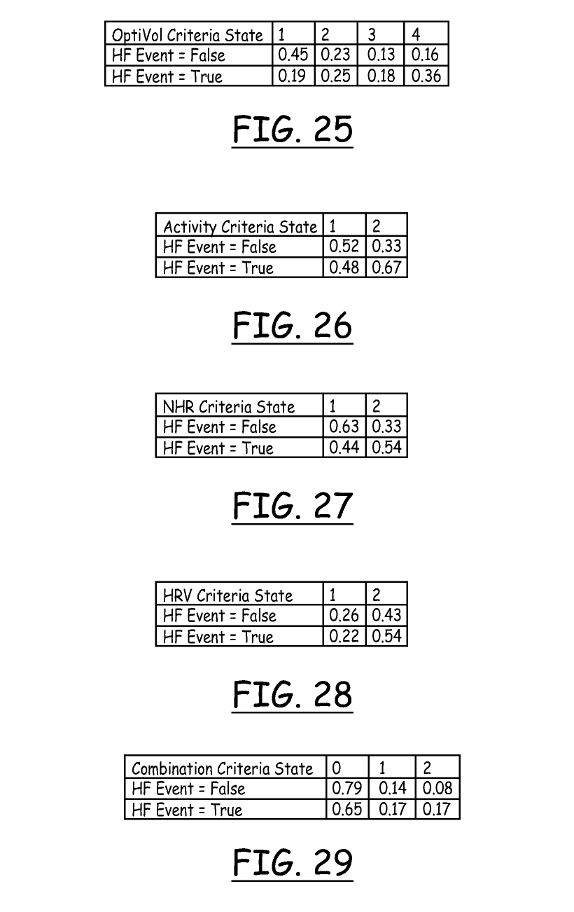

Likelihood tables were generated from data from the OFISSER, CRT patients in CONNECT study, and the “Observation Only” patients in the Italian Clinical Services data as described in earlier section. The likelihood tables for the device parameters are shown in the tables of FIGS. 25-31.

FIG. 25 s a likelihood table for the Opti-Vol criteria states.

FIG. 26 is a likelihood table for the Activity criteria states.

FIG. 27 is a likelihood table for the NHR criteria states.

FIG. 28 is a likelihood table for the HRV criteria states.

FIG. 29 is a likelihood table for the Combination criteria states.

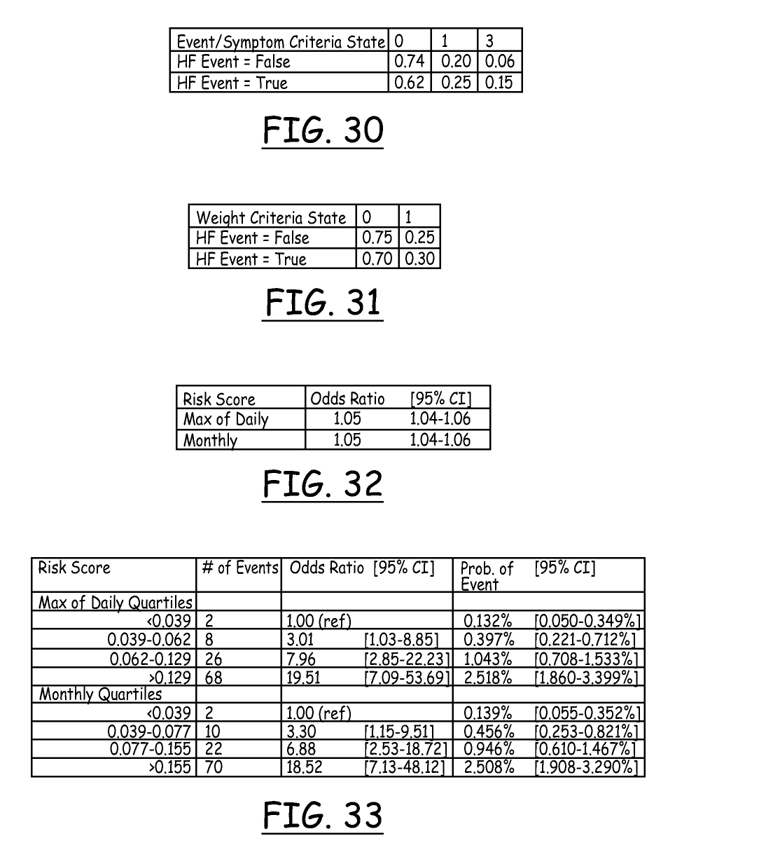

FIG. 30 is a likelihood table for the Event/Symptom criteria states.

FIG. 31 is a likelihood table for the Weight criteria states.

Note that the table of FIG. 29 has a criteria state of “0” that has not been defined in the methods. The criteria state of 0 is treated as a criteria state of “−1” and is never used to generate posterior probability; however the probability of the state is defined for the BBN initialization.

The likelihood tables for the clinical variables (FIGS. 30 and 31) were educated guesses based on historic data. Weight, blood pressure, and medication data was not available for the development dataset and hence has not been used to generate the results in this report. The blood pressure and the medication likelihood tables can be defined similar to the weight likelihood table shown in FIG. 31.

### Development Dataset Results

The results of the GEE model for the entire development set of 921 patients with 9790 monthly evaluations with 104 monthly evaluations having at least one event in the following month is shown in the table of FIG. 32. The results are reported including all the available parameters (device plus baseline plus clinical data). Both types of risk score computations are reported. The Odds Ratio in this case can be interpreted as follows: every percentage increase of the Risk Score corresponds to a certain increased risk of an HF event. For example, for all the parameters (i.e. device plus baseline plus clinicals) the Odds ratio is 1.05, which means that for every percentage increase in the HF risk score the probability of HF event in the next 30 days increase by 5%. The confidence interval from [1.041.06] indicates that there is a very statistically significant relationship as it does not include the value of 1.0.

The risk score was divided into quartiles and the raw number of events in each group, odds ratio between the different groups and the probability of event (adjusted for repeated observations) is shown in the table of FIG. 33 for the entire development dataset. All the parameters (i.e. device plus baseline plus clinical variables) were used to generate the results.

If the risk score is in one of the quartile ranges, then the Odds Ratio states the increased risk of HF event when compared to that in the lowest quartile (which acts as reference). Thus, if the “Max of Daily” HF risk score is>0.129 then the risk of HF hospitalization is 19.51 times the risk of hospitalization if the risk score was<0.039. Note that the Odds ratio increases as the risk score decreases into a higher quartile as indicated by the GEE model results in the previous table. Note that the probability of event is very low due to the low clinical monthly event rate for HF hospitalizations for an individual patient. However, the risk of the HF event increases significantly as the HF risk score increases.

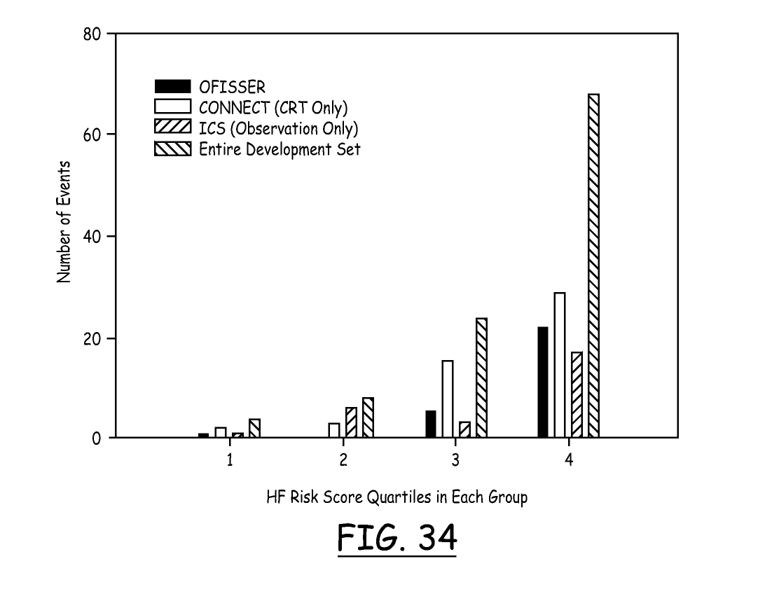

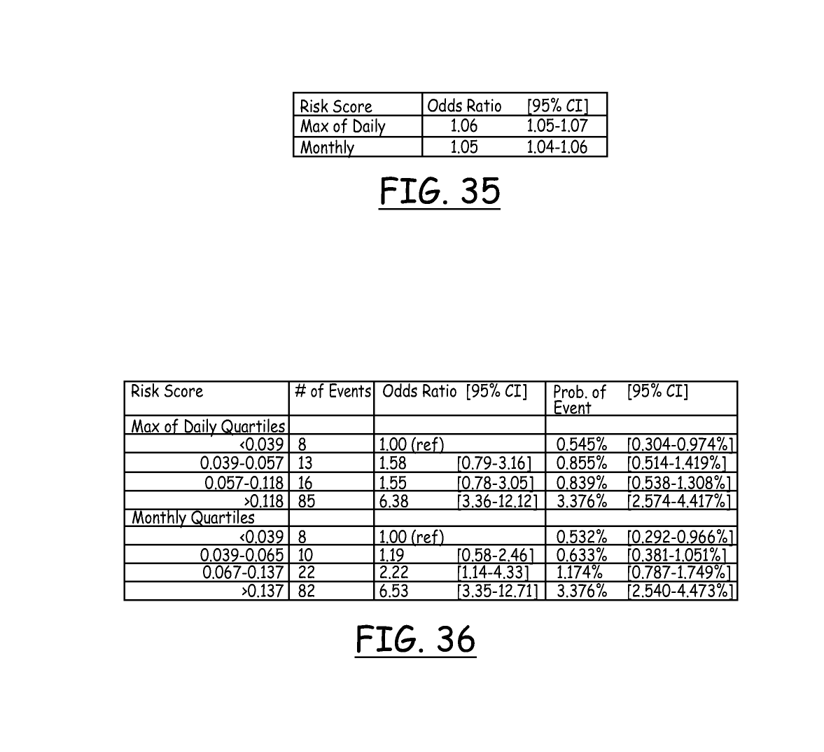

The distribution of the raw number of events in each of the quartiles of HF risk score (max of daily) for the development set (and each study in the development set) is shown in FIG. 34. Each group had its own boundaries for the quartiles. For the entire development set, the raw number of events is detailed in the table of FIG. 35. There were 68 events in the high risk quartile versus 4 in the low risk quartile. Thus, the risk score is useful in stratifying patients at higher risk at follow-up.

### Validation Dataset Results

The results of the GEE model for the entire validation set of 784 patients with 8149 monthly evaluations with 122 monthly evaluations having at least one event in the following month is shown in the table of FIG. 35. The results are reported including all the available parameters (device plus baseline plus clinical data). Both types of risk score computations are reported. The Odds Ratio in this case can be interpreted as follows: the HF risk score (max of daily) for all the parameters (i.e. device plus baseline plus clinical parameters) the Odds ratio is 1.06, which means that for every percentage increase in the HF risk score the probability of HF event in the next 30 days increase by 6%. The confidence interval from [1.05-1.07] indicates that there is a highly statistically significant relationship as it does not include the value of 1.0.

The table of FIG. 35 sets forth the HF Risk Score Logistic Regression results for the entire validation set.

The risk score was divided into quartiles and the number of raw events in each group, the odds ratio between the different groups (adjusted for repeated observations) and the probability of event (adjusted for repeated observations) is shown in Table 20 for the entire development dataset. All the parameters (i.e. device plus baseline plus clinical parameters) were used to generate the results.

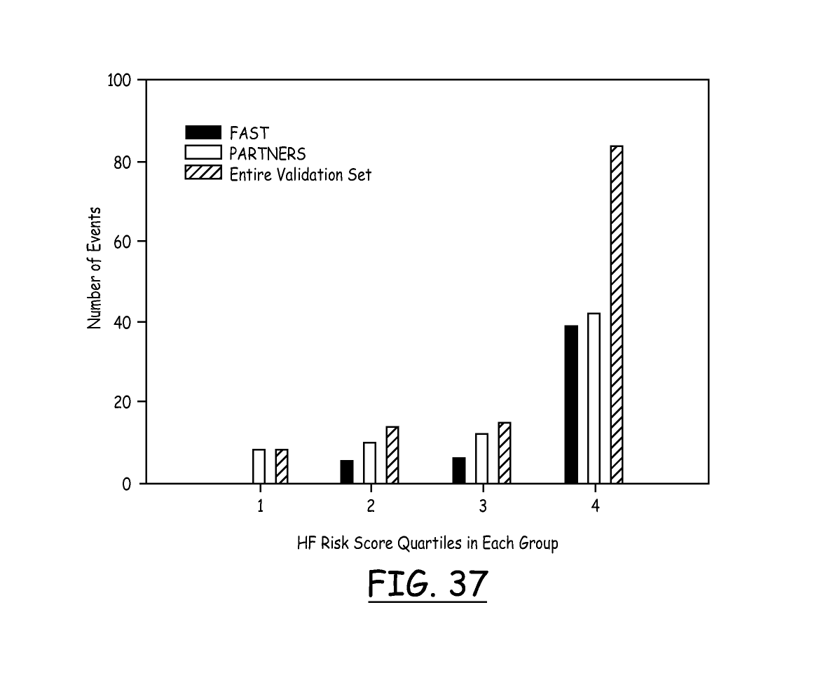

The table of FIG. 37 sets forth the HF Risk Scores divided into quartiles for the validation set.

If the risk score is in one of the quartile ranges, then the Odds Ratio states the increased risk of HF event when compared to that in the lowest quartile (which acts as reference). Thus, if the “Max of Daily” HF risk score is>0.118 then the risk of HF hospitalization is 6.38 times the risk of hospitalization if the risk score was<0.039. Note that the odds ratio goes up as the risk score falls into a higher quartile as indicated by the logistic regression results in the previous table. Note that the probability of event is very low due to the low event rate for HF hospitalizations. However, the risk of the HF event goes up significantly as the HF risk score increases. The distribution of the raw number of events in each of the quartiles of HF risk score (max of daily) for the validation set (and each study in the validation set) is shown in FIG. 13. Each group had its own boundaries for the quartiles.

For the entire validation set the raw number of events is detailed in the table of FIG. 36. There were 85 events in the high risk quartile versus 8 in the low risk quartile showing the value of the risk score in stratifying patients at higher risk at follow-up. The risk score performs much better in the FAST study where physicians were blinded to the OptiVol data. The ratio of the raw events between the highest and the lowest quartile is 10.6 (85/8), whereas the overall odds ratio after adjusting for repeated observations is only 6.38. This happens because the PARTNERS study has a larger number of patients with short duration of follow-up, whereas the FAST study had a smaller number of patients with a very long duration of follow-up. Although the number of events was pretty similar between the two studies, but the statistical adjustment process weighs the PARTNERS study more than the FAST study. It should be noted that all of the events in the lowest quartile came from the PARTNERS study, indicating that the FAST study performed very well with respect to the risk score.

The Bayesian approach to an integrated heart failure diagnostic (HF Risk Score) provided by the present invention thus successfully generates a continuous absolute HF risk score that appropriately identified time periods with clinically and statistically significantly higher relative risk for near term HF clinical events. Conversely, the index also identifies a group at relatively low risk for events. This is superior to a single parameter approach, for example OptiVol, or to an (apples and apples) Bayesian approach using only a single parameter.

The HF risk score provided by the present invention may be displayed to the physician in any useful format. However, the inventors have developed a preferred display methodology which is believed to incorporate the HF risk score into a diagnostic record in a particularly useful and easy to understand fashion. A preferred example of such a display methodology is described below.

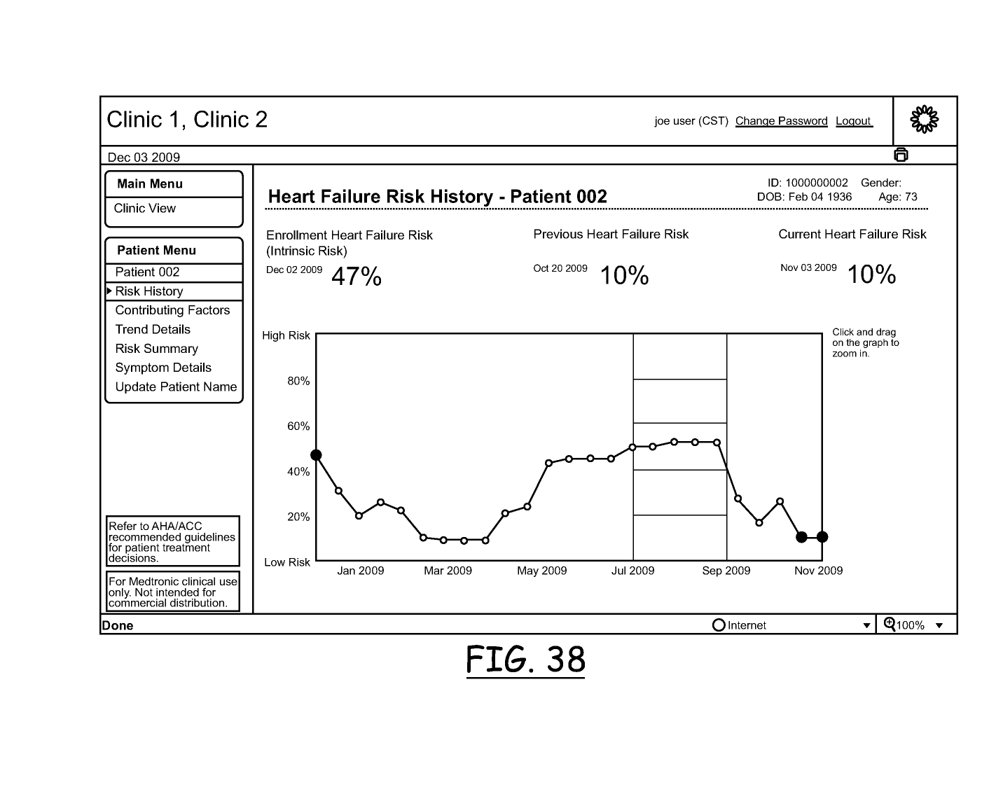

The pages illustrated in FIGS. 38-41 should be understood as displays provided by the programmer 24 or by a physicians computing device 316, as illustrated in FIG. 4. The diagnostic data reflected in the display is obtained and distributed as described above. The information displayed and the linkages between the pages are described below.

The Patient's Risk History page, shown in FIG. 38, presents a trend graph of the Heart Failure Risk Scores along with three Risk Scores at significant points in time. The numerically displayed Risk Scores are the (1) Enrollment or Intrinsic Risk, the (2) Previous Risk, and the (3) Current Risk.

The Enrollment or Intrinsic Risk is the patient's calculated Risk Score at the beginning of the study. In other words, this is the patient's intrinsic risk at the time of enrollment. This is derived using the patient's medical history only. The Previous Risk is the patient's previously calculated Risk Score using data from the next most current device transmission received. The Current Risk is the patient's most current calculated Risk Score using data from the last device transmission received.

The Risk History Trend Graph plots the Risk Scores calculated at their associated device transmission date with a circle as the data point. The three significant Risk Score points are preferably highlighted as follows; The Enrollment or Intrinsic Risk Score is plotted with a black point; the Previous Risk Score is plotted with a purple point; and the Current Risk Score is plotted with a blue point.

Rolling over one of the three significant Risk Score points will preferably also include the appropriate text label of “Enrollment”, or “Previous”, or “Current”. When the cursor is moved over a data point, a popup “tip” will be displayed with the Risk Score and date of calculation. Holding down the left mouse button and dragging over a region on the graph then releasing the left mouse button will preferably zoom in on that particular time interval region on the graph. After the graph has been redisplayed a “Reset Zoom” link will preferably appear to the right of the graph. Selecting this link will preferably return the graph to it original time interval.

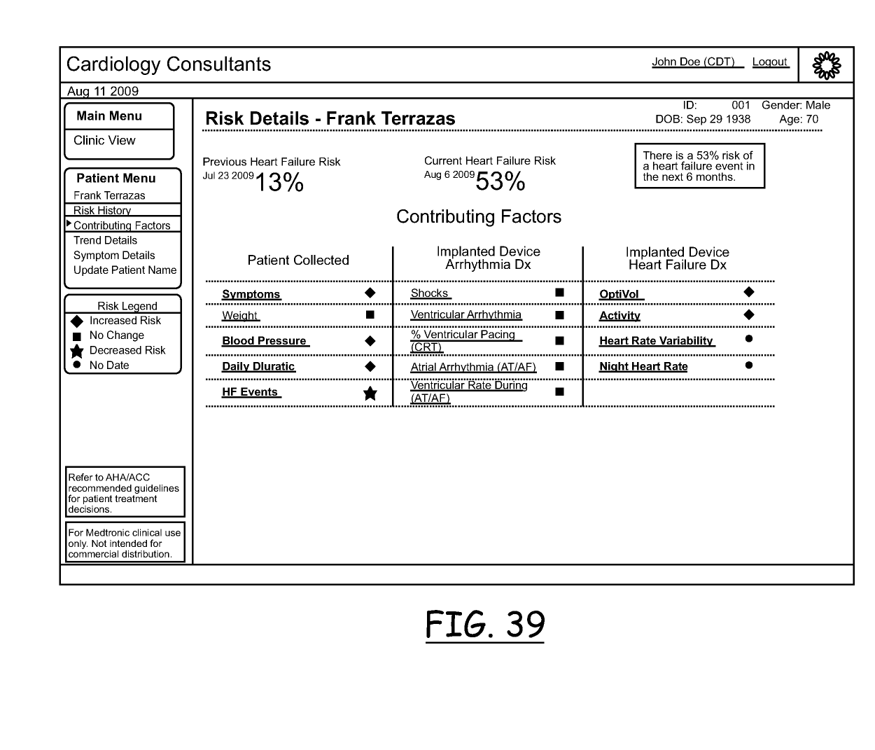

The Patient's Contributing Factor page, shown in FIG. 39, presents a tabulation of the change in the risk from the Previous Risk Score to the Current Risk Score for each Risk Score contributing factor along with a summary highlighting the Previous and Current Risk Scores.

The Previous Risk is the patient's previously calculated Risk Score using data from the next most current device transmission received, as discussed above. The Current Risk is again the patient's most current calculated Risk Score using data from the most current device transmission received.

The Contributing Factor Table shows the factors contributing to the Current Heart Failure Risk Score in three columns, grouped by their data source. Each Contributing Factor listed in the table is preferably a page link to respective detail graph in the Trend Details Page, discussed below. Selecting any of the factor links will preferably display the Trend Details Page and automatically scroll the page so that the selected factor's graph is visible.

### Patient Collected Factors

The data for the factors listed in this group primarily originate from the patient's Health Monitor and from the clinic entered Case Report Forms.

### Arrhythmia Diagnostics (Dx) Factors

The data for the factors listed in this group originate from the patient's implanted device collected arrhythmia metrics obtained from device transmissions.

### Heart Failure Diagnostics (Dx) Factors

The data for the factors listed in this group originate from the patient's implanted device collected heart failure metrics obtained from device transmissions.

The Risk Legend shows the symbols used in the Contributing Factors table to indicate the change in the risk from the Previous Risk Score to the Current Risk Score for a contributing factor, as follows:

“Increased Risk”. The factor contributed to an increased risk.
“No Change”. The factor contribution to the risk has remained the same. Note: This does NOT imply that this factor did not contribute to the risk, but rather that its level of contribution has not changed.
“Decreased Risk”. The factor contributed to a decreased risk.
“No Data”. Insufficient data was available to determine the contribution.

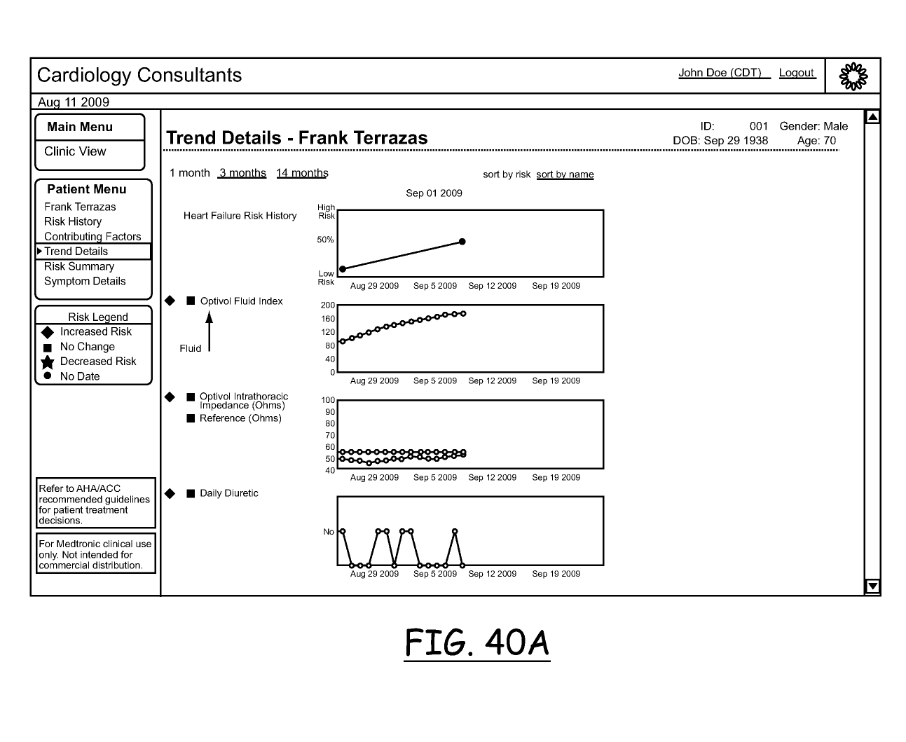

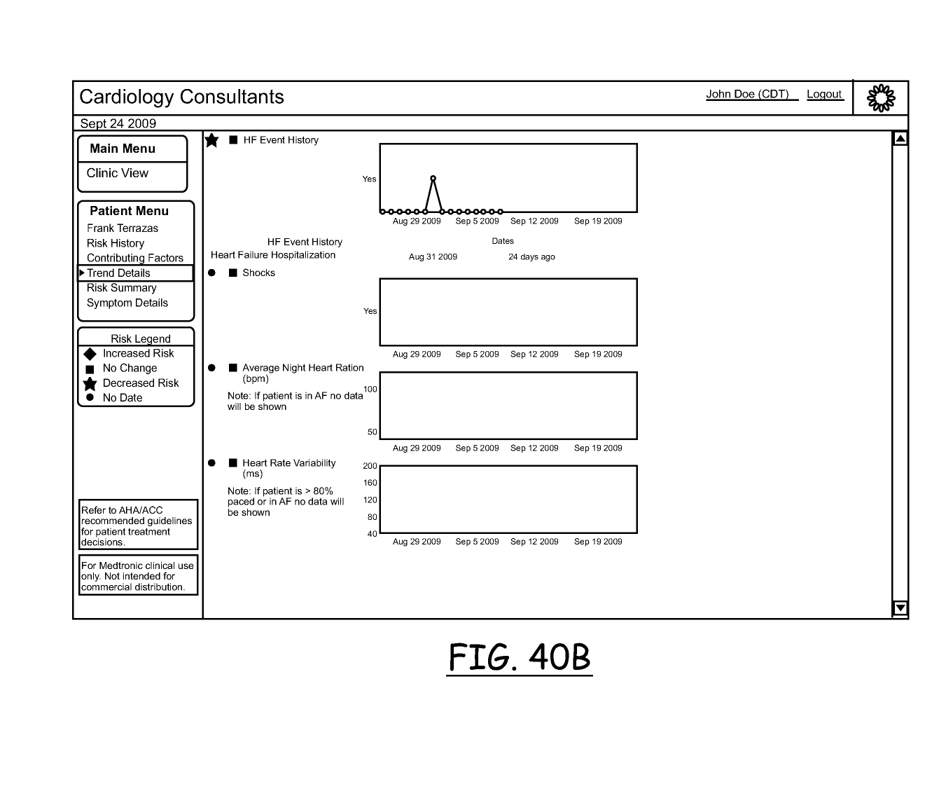

The Patient's Trend Details Page, shown in FIGS. 40A and 40B, shows a trend graph of the Heart Failure Risk Score and a trend graph and/or table for each of the Risk Score Contributing Factors. The Heart Failure Risk History trend graph will preferably be displayed at the top of the page. The order of the remaining graphs/tables will preferably be determined by the page's sorting order and which initially is descending sorted by the Contributing Factor's change in risk.

The Heart Failure Risk History is a trend graph of the computed Heart Failure Risk Scores along with three Risk Scores at significant points in time. This graph is the same graph displayed in the Patient's Risk History Page (FIG. 38) and is displayed at the top of the page. Each of the Risk Score Contributing Factors will preferably have an individual trend graph plotting all of the factor's data points. Along with each plotted trend graph will be a symbol indicating the Risk change associated with that Contributing Factor and a symbol legend of the plotted points.

A Heart Failure (HF) Event History table, as outlined below, may also be provided, chronologically listing the reason, date, and time since the event for the patient's HF related Health Care Utilization (HCU) events. The Heart failure Event History Table is preferably associated with the Heart Failure Trend details page as discussed below. Possible HCU event reasons (if multiple HCU instances occur associated the clinical event, preferably only the most significant HF event will be listed) can be on of the following:

### Pre-Enrollment Events

### “Pre-enrollment HF Hospitalization”

### “Pre-enrollment Emergency Department Visit”

Post Enrollment (in order or significance)
“Hospitalization with oral diuretic dosage doubling in 1 day”
“Hospitalization with IV diuretics for treatment of congestive HF”
“Emergency Dept visit with IV diuretics for treatment of congestive HF”
“Clinic visit with IV diuretics for treatment of congestive HF”

The trend graph zooming function for the Trend Details page preferably operates as described in the Risk History Trend Graph. Each trend graph may be zoomed independently. Note: The date shown in the Graph Indicator Line label box is the date of the unzoomed trend graphs, not the date in a zoomed graph. When the cursor is moved over a data point, a popup “tip” will be preferably displayed with the value and date of that data point.

The Risk Legend shows the symbols used to indicate the following types of changes in the risk from the Previous Risk Score to the Current Risk Score for a contributing factor:

“Increased Risk” . The factor contributed to an increased risk.
“No Change” . The factor contribution to the risk has remained the same. Note: This does not imply that this factor did not contribute to the risk, but rather that
“Decreased Risk” . The factor contributed to a decreased risk.
“No Data” . Insufficient data was available to determine the contribution.

The order of the graphs can preferably be arranged either by descending contributing factor risk or in alphabetical order by selecting the respective sort link. Irrespective of the sort order chosen, the Heart Failure Risk History graph will preferably always be displayed at the top of the page.

The graphs on the Trend Details page can preferably be viewed in a several time intervals: the previous 1 month, the previous 3 months, and the previous 14 months. Selecting any one of these links will rescale all graphs on the page to the newly selected time interval.

The Trend Details Page preferably also provides the user with a Graph Indicator Line as illustrated as an aid for aligning points of interest across all graphs. The Graph Indicator Line is activated by selecting a “Drag Me” label box with the left mouse button and dragging it horizontally into the page area containing the trend graphs as shown in FIG. 9.

Upon the label box entering the trend graph area, the following will preferably occur: 1. A vertical red Indicator Line will be drawn from the label box and extending through all graphs; 2. As the label box enters the plotted data area, the “Drag Me” text will be replaced with the trend graph date intersected by the vertical red line; 3. The date in the label box will be continuously updated as the Indicator Line is moved; 4. The Indicator Line will remain in position when the mouse button is released; and 5. Selecting and dragging the Graph Indicator Line label box outside the graph area will cause the “Drag Me” label to reappear. Note: The date shown in the Graph Indicator Line label box is the preferably date of the unzoomed trend graphs. If any individual trend graphs have been zoomed, the Graph Indicator Line date will not indicate the date in those zoomed graphs.

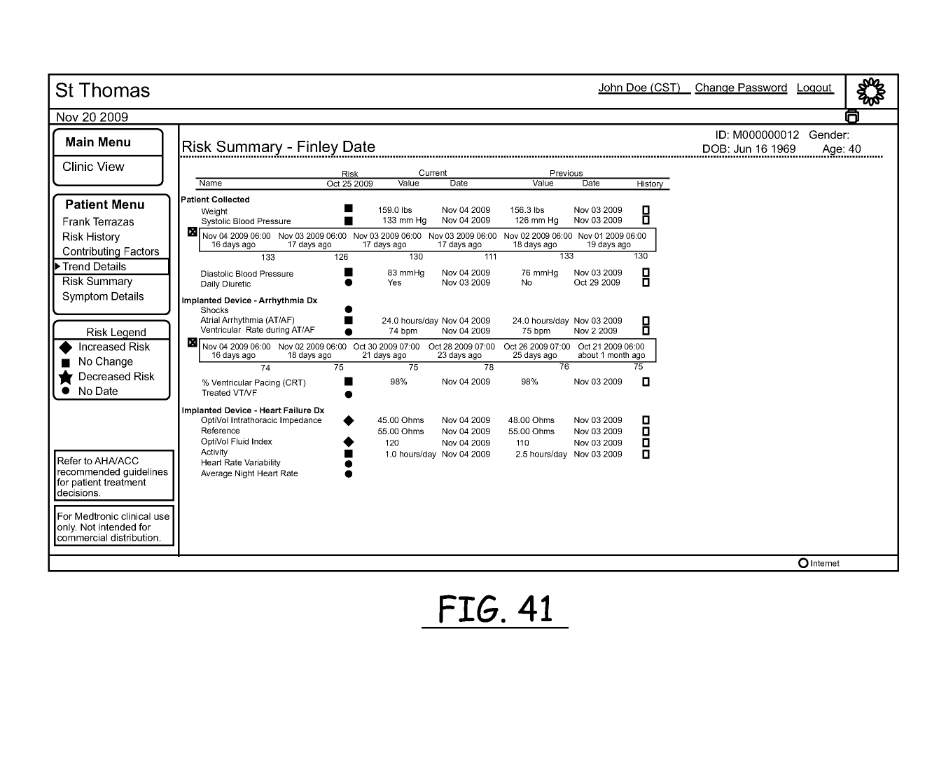

The Risk Summary Page, shown in FIG. 41, shows a tabular summary of the Contributing Factor risk contributions and their recent values. A popup history table, using the most recent 6 values, is preferably available also for each factor.

The Risk Summary table preferably contains the following columns of information for each Contributing Factor of the Risk Score:

### Name

The name of the Risk Score Contributing Factor
Risk The Risk Change symbol representing the change in the factor's risk Score contribution
Current: The value and value's date of the Contributing Factor used in the Current Risk Score calculation.
Previous: The value and value's date of the Contributing Factor used in the Previous Risk Score calculation.
History: An icon link to invoke the Contributing Factor's popup history table, using the most recent 6 values.

A history table for an individual factor is preferably available by selecting the Contributing Factor's History icon . This will in turn cause a table to popup listing the last 6 factor values to be displayed.

An associated Patient's Symptom Details Page as follows may also be provided. Such a Symptom Details Page may show a tabulation of the answers to weekly questions asked of the patient. The questions may be grouped into 5 categories with up to 6 weeks of answers displayable at a time, one answer per column. Patients are typically asked only one question from each category each week. The most recent answer sets will be initially displayed. Shown in the Symptom Details Page

The combination of the HF Risk Score determination methodology with the display methodology described above is believed to provide a substantial enhancement to the ability of physicians to monitor and treat heart failure patients.

While a detailed description of the preferred embodiments of the invention has been provided, it is recognized that numerous modifications or variations may be made to the specifically disclosed embodiments of the present invention. It is intended, therefore, in the following claims to cover all such changes and modifications as may fall within the true scope of the invention. For example, the use of a Bayesian belief system based methodology as described herein to provide other predictive diagnostic indexes, based upon alternate data inputs is also believed useful. In particular, the present invention is believed equally useful in the context of other known fluid measurement methodologies known to the art and other cardiac rhythm analysis methodologies known to the art.

1. A method comprising:
monitoring at multiple primary diagnostic parameters associated with worsening heart failure, including receiving at least one of said parameters from an implanted medical device;
deriving an index of the likelihood of worsening heart failure from the multiple diagnostic parameters by using a Bayesian approach; and
displaying the derived index.

2. The method of claim 1, wherein the received parameter from the implanted medical device comprises intrathoracic impedance.

3. The method of claim 1, further comprising generating an alert in response to detecting worsening heart failure in the patient.

4. The method of claim 1, further comprising automatically modifying a therapy delivered to the patient in response to detecting worsening heart failure in the patient.

5. The method of claim 16, wherein the therapy comprises at least one of a substance delivered by an implantable pump, cardiac resynchronization therapy, refractory period stimulation, or cardiac potentiation therapy.

6. A system comprising:
at least one implantable sensor;
a processor that:
monitors multiple parameters associated with worsening heart failure, including at least one parameter received from the sensor from an implanted medical device;
derives an index of the likelihood of worsening heart failure from the multiple diagnostic parameters by using a Bayesian approach; and
a display responsive to the processor and which displays the derived index.

7. The system of claim 6, wherein the at least one sensor comprises a plurality of electrodes, and the primary diagnostic parameter comprises intrathoracic impedance.

8. The system of claim 6, wherein the at least one sensor comprises at least one electrode located on an implantable medical lead coupled to an implantable medical device.

9. The system of claim 6, wherein the at least one sensor is at least one of within or coupled to an implantable medical device, and the processor comprises a processor of the implantable medical device.

10. The system of claim 9, further comprising an external device that wirelessly communicates with the implantable medical device, wherein the processor monitors the at least one parameter based on information input by a user via the external device.

11. The system of claim 9, wherein the processor provides an alert to a user in response to detecting worsening heart failure in the patient.

12. The system of claim 9, further comprising an implantable medical device that delivers a therapy to the patient, wherein the processor automatically modifies the therapy in response to detecting worsening heart failure in the patient.

13. The system of claim 12, wherein the therapy comprises at least one of a substance delivered by an implantable pump, cardiac resynchronization therapy, refractory period stimulation, or cardiac potentiation therapy.

14. A non-transitory computer-readable medium comprising instructions that cause a processor to:
monitor at least multiple diagnostic parameters associated with worsening heart failure including at least one parameter received from an implanted medical device;
derive an index of the likelihood of heart failure from the multiple diagnostic parameters by using a Bayesian approach; and
display the derived index.

15. A system comprising:
means for monitoring multiple diagnostic parameters indicative of worsening heart failure including at least one diagnostic parameter received from an implanted medical device;
means for deriving an index value over time using a Bayesian approach based on the diagnostic parameters, wherein the index indicates a likelihood of worsening heart failure of the patient;
means for displaying the derived index.
